(19) United States
(12) Patent Application Publication
(10) Pub. No.: US 2019/0153552 A1
(43) Pub. Date: May 23, 2019

(54) HPV SCREENING PLATFORM

(71) Applicant: Mayo Foundation for Medical Education and Research, Rochester, MN (US)

(72) Inventors: Marina R. Walther-Antonio, Rochester, MN (US); Yuguang Liu, Rochester, MN (US)

(73) Assignee: Mayo Foundation for Medical Education and Research, Rochester, MN (US)

(21) Appl. No.: 16/300,398

(22) PCT Filed: May 10, 2017
(86) PCT No.: PCT/US2017/031870

§ 371 Date: Nov. 9, 2018

Related U.S. Application Data
(60) Provisional application No. 62/334,922, filed on May 11, 2016.

(51) Int. Cl. C12Q 1/70; G01N 21/78; C12Q 1/6834

(52) U.S. Cl. CPC C12Q 1/708; G01N 21/78; C12Q 1/6834; C12Q 2563/125; G01N 2333/01; C12Q 2537/143; C12Q 2537/125

(57) ABSTRACT

Materials and methods are provided herein for determining if a subject has, or is at risk of developing, a clinical condition (e.g., cervical cancer). For example, this document provides a cost-effective self-test for determining whether a biological fluid from a subject contains a high-risk HPV strain, and a method for use of the self-test.

### CROSS-REFERENCE TO RELATED APPLICATIONS

This application claims priority from U.S. Provisional Application Ser. No. 62/334,922, filed May 11, 2016.

### TECHNICAL FIELD

This document relates to materials and methods for determining if a subject has, or is at risk of developing, a clinical condition that can be detected based on the presence of particular markers. For example, this document relates to a cost-effective self-test for determining whether a biological fluid from the subject contains a nucleic acid or polypeptide marker for a virus (e.g., a high-risk HPV strain that may indicate cervical cancer) or a bacterium (e.g., Escherichia or Salmonella).

### BACKGROUND

A large number of diagnostic assays for various clinical conditions, including assays that involve the use of polymerase chain reaction (PCR) or enzyme-linked immunosorbent assays (ELISAs), can take 24 to 48 hours or even longer to produce results, which can negatively impact urgent decision making in particularly vulnerable patients.

For example, cervical cancer often is asymptomatic at an early and curative stage. Readily available and reliable screening is important, particularly in populations without access to human papillomavirus (HPV) vaccination. The performance of a single HPV testing round has been associated with a significant reduction in the number of advanced cervical cancers, and HPV testing has been demonstrated to be more sensitive for detecting cancerous and precancerous microlesions than visual inspection with acetic acid and cytologic testing (Sankaranarayanan et al., N Engl J Med 360(14):1385-1394, 2009). Approaches such as the CAREHPV® test (Qiagen Inc.; Valencia, Calif.) offer a sensitivity of 90% and specificity of 84% (Qiao et al., The Lancet Oncol 9(10):929-936, 2008) for detecting pre-malignant or malignant lesions, but the cost, processing time, and need for laboratory facilities may be prohibitive for using such approaches in low-resource settings. Cervical cancer screening in low-resource areas also can be challenged by disease priority, social stigma, and local healthcare systems. For example, screening may be marginalized compared to acute medical problems, there may be social stigma related to HPV infection or even visiting a gynecologist in conservative communities, and/or the local healthcare infrastructure may not have the resources to support a women's health program.

### SUMMARY

The present document is based, at least in part, on the development of a point-of-care testing method that can expedite screening capabilities while providing accurate, rapid, and affordable diagnoses, which can lead to timely and informed medical intervention. The materials and methods described herein relate to the use of in-vial dry reagent storage, which can allow for rapid “mix-and-read” diagnosis with the naked eye. The materials and methods can be used for detecting various clinical conditions or viral and bacterial infections, indicating, for example, that a subject is infected with HPV or influenza virus, is a carrier of Streptococcus pneumoniae, or contains circulating cardiac endothelial cells (CECs), which can indicate cardiac emergency. The materials and methods provided herein can eliminate delays in diagnostic testing since there is no need to transport samples to a laboratory setting, and no need for specialized personnel to perform the required assays and interpret the results.

In some embodiments, for example, this document provides an inexpensive, reliable self-test for high-risk HPV. The screening test can be used to identify subjects (e.g., human females) having a high-risk carrier status, while minimizing barriers and providing an opportunity for early intervention before disease leads to significant morbidity and mortality. The self-test can be cost effective, easily distributed, and can be administered in the privacy of a user's home. This may lead to earlier detection of HPV, resulting in clinical follow up in a more timely manner.

In a first aspect, this document features a kit containing (a) a receptacle for receiving a biological fluid sample, the receptacle having a first nucleic acid reversibly attached to a first interior surface and a second nucleic acid immobilized on a second interior surface, wherein the first nucleic acid is complementary to a first nucleic acid sequence of a selected marker, and wherein the second nucleic acid is complementary to a second nucleic acid sequence of the marker; and (b) a substrate having a first portion with an integrated positive control result and a second portion for receiving a test fluid. The receptacle can be a glass vial (e.g., a glass vial with a volume of 10 to 20 mL). The first nucleic acid can be dry-stored on the first interior surface. The second nucleic acid can be coupled to an agarose film on the second interior surface. The first nucleic acid can be coupled to horseradish peroxidase (HRP). The marker can be from one or more high-risk human papillomavirus (HPV) strains (e.g., one or more of HPV 16, HPV 18, HPV 31, HPV 33, HPV 35, HPV 39, HPV 45, HPV 51, HPV 52, HPV 56, HPV 58, HPV 59, and HPV 68). The substrate can include a paper strip.

In another aspect, this document features a kit containing (a) a receptacle for receiving a biological fluid sample, the receptacle having a nucleic acid reversibly attached to a first interior surface and a reagent reversibly attached to a second interior surface, wherein the nucleic acid is complementary to a nucleic acid sequence of a selected marker, and wherein the reagent binds specifically to a complex formed when the reversibly attached nucleic acid hybridizes to the nucleic acid sequence of the selected marker; and (b) a substrate having a first portion with an integrated positive control result and a second portion for receiving a test fluid. The receptacle can be a glass vial (e.g., a glass vial with volume of 10 to 20 mL). The reversibly attached nucleic acid can be dry-stored on the first interior surface. The reagent can be coupled to an agarose film on the second interior surface. The reagent can be hemin. The marker can be from one or more high-risk HPV strains (e.g., one or more of HPV 16, HPV 18, HPV 31, HPV 33, HPV 35, HPV 39, HPV 45, HPV 51, HPV 52, HPV 56, HPV 58, HPV 59, and HPV 68). The substrate can include a paper strip.

In another aspect, this document features a method for determining that a biological fluid contains a selected marker, where the method includes (a) providing a receptacle for receiving the biological fluid, the receptacle having a first nucleic acid reversibly attached to a first interior surface and a second nucleic acid immobilized on a second interior surface, where the first nucleic acid is complementary to a nucleic acid sequence of the marker and is labeled with a means for visual detection, and where the second nucleic acid is complementary to a second nucleic acid sequence of the marker; (b) placing a sample of the biological fluid into the receptacle, such that the first nucleic acid is released from the first interior surface, and the first and second nucleic acids hybridize to the first and second complementary nucleic acid sequences of the marker, such that the first nucleic acid becomes attached to the second interior surface via the marker and the second nucleic acid; (c) removing the biological fluid from the receptacle; (d) washing the interior of the receptacle to remove residual biological fluid; (e) placing a substrate that interacts with the means for visual detection into the receptacle, such that the means for visual detection can act on the substrate to generate a signal; and (f) visually inspecting the vial containing the substrate, or a sample of the substrate, to determine that the signal is present, thus indicating the presence of the high-risk HPV strain in the biological fluid. The receptacle can be a glass vial (e.g., a glass vial with a volume of 10 or 20 mL). The first nucleic acid can have been dry-stored on the first interior surface prior to providing the receptacle. The second nucleic acid can have been coupled to an agarose film on the second interior surface prior to providing the receptacle. The means for visual detection can be HRP. The substrate can be tetramethylbenzidine (TMB). The marker can be from one or more high-risk human HPV strains (e.g., one or more of HPV 16, HPV 18, HPV 31, HPV 33, HPV 35, HPV 39, HPV 45, HPV 51, HPV 52, HPV 56, HPV 58, HPV 59, and HPV 68). The biological fluid can include urine or vaginal fluid.

In yet another aspect, this document features a method for determining that a biological fluid contains a selected marker, where the method includes (a) providing a receptacle for receiving the biological fluid, the receptacle having a nucleic acid reversibly attached to a first interior surface and a reagent reversibly attached to a second interior surface, where the nucleic acid is complementary to a nucleic acid sequence from the marker, and where the reagent binds specifically to a complex formed when the reversibly attached nucleic acid hybridizes to the nucleic acid sequence of the marker; (b) placing a sample of the biological fluid into the receptacle, such that (i) the nucleic acid is released from the first interior surface and binds to the complementary nucleic acid sequence of the marker to form a complex, and (ii) the reagent binds to the complex to generate a signal; (c) placing in the receptacle a substrate that interacts with the complex; and (d) inspecting the vial, or a sample from the vial, to determine that the signal is present, thus indicating the presence of the marker in the biological fluid. The receptacle can be a glass vial (e.g., a glass vial with a volume of 10 or 20 mL). The nucleic acid can have been dry-stored on the first interior surface prior to providing the receptacle. The reagent can have been dry-stored on the second interior surface prior to providing the receptacle. The reagent can be hemin. The substrate can be TMB. The marker can be from one or more high-risk human HPV strains (e.g., one or more of HPV 16, HPV 18, HPV 31, HPV 33, HPV 35, HPV 39, HPV 45, HPV 51, HPV 52, HPV 56, HPV 58, HPV 59, and HPV 68). The biological fluid can include urine or vaginal fluid.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic depicting the components and steps in a vial-based self-test for detection of HPV in a fluid test sample.

FIG. 2 is a schematic depicting the components and steps in a vial-based self-test for using a DNAzyme reagent to detect a target molecule in a fluid test sample.

### DETAILED DESCRIPTION

This document provides diagnostic tests that can be applied to any sample of bodily fluid (e.g., urine, sputum, blood, serum, or cerebrospinal fluid) suspected of containing a particular marker molecule (e.g., a nucleic acid such as an RNA or DNA, or a polypeptide or protein). This document also provides kits containing the tests, as well as methods of using the test materials to detect markers from selected targets, and to determine whether cells containing a selected marker are present in a biological sample. Markers that may be detected using these materials and methods include, without limitation, nucleic acids (e.g., mutant or aberrantly expressed nucleic acids that provide a signature for a disease), peptides, polypeptides, antibodies or antibody fragments, virus particles, and bacteria.

In some cases, an existing PCR or ELISA-based test can be adapted to the platform described herein. The platform can provide a high clinical value for detecting acute illness in patients who would benefit from real-time testing and immediate administration of targeted treatment, although the platform also can be applied to any chronic condition for which there is, for example, a nucleic acid or protein marker. As compared to standard diagnostic procedures, the materials and methods provided herein typically are faster (providing real-time results) and more affordable, and allow for the possibility of self-administration.

In some embodiments, the assay platform provided herein can be used as a self-test to identify high-risk HPV carriers, in methods for determining whether a subject is a carrier of high-risk HPV. In women, high-risk types of HPV (such as types 16, 18, 31, and 45) can cause changes in the cells of the cervix that can be seen as abnormalities on a Pap test. Abnormal cervical cell changes may resolve on their own without treatment, but some untreated cervical cell changes can progress to serious abnormalities and may lead to cervical cancer over time if they are not treated. The screening tests described herein can provide for rapid and easy detection of the high-risk HPV in carriers. The tests can take advantage of target strain labeling and capturing capabilities in a receptacle (e.g., a glass vial), based on complementary HPV nucleic acid immobilization and dry reagent storage. The test can be read in real-time, without requiring laboratory facilities or personnel for the processing of results, which can address some of the main challenges with implementation in low-resource communities. The test is aimed at matching the clinical standard for HPV testing reliability, with a user-perceived level of difficulty for use and interpretation that is comparable to standard home pregnancy test use and reading.

The screening kits provided herein include a receptacle (e.g., a glass vial) as a nucleic acid diagnostic platform that can be used as a self-test and read in real-time by a user, with minimal instruction. The described approach for nucleic acid detection can include pre-immobilization of nucleic acid (e.g., RNA or DNA) sequences that are complementary to high-risk HPV nucleic acid sequences and dry storage of reagents in the receptacle that allow for labeling and capture of target strains, and produce colorimetric results interpretable by the naked eye. In some embodiments of the test methods described herein, nucleic acids complementary to marker sequences (e.g., from high-risk HPV strains) can be modified with horseradish peroxidase (HRP) and dry-stored in vials. When the complementary nucleic acids are contacted with a fluid containing an HPV marker nucleic acid (e.g., RNA or DNA) and a tetramethylbenzidine (TMB) substrate, the fluid can turn blue to indicate the presence of a high-risk HPV strain.

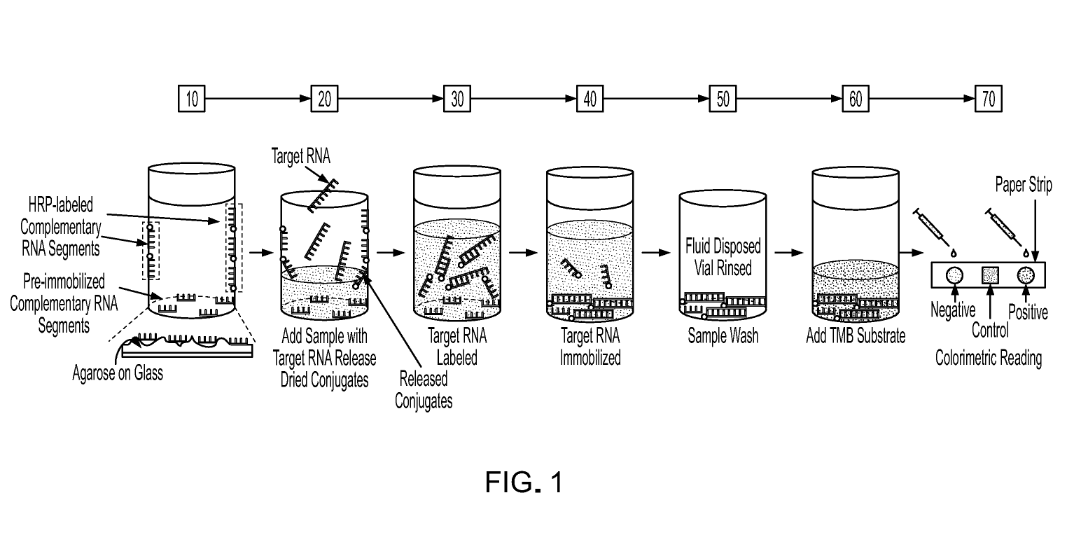

FIG. 1 depicts the components and methodology of an exemplary HPV screening test as provided herein. The test relies on labeling and capture of target nucleic acid from a biological fluid sample (e.g., vaginal fluid or urine) in a receptacle (e.g., a 10 mL glass vial), followed by a wash step to remove the supernatant.

In the depicted method, one or more first nucleic acid (e.g., RNA) sequences that are complementary to HPV nucleic acid sequences (e.g., RNA sequences from one or more high-risk HPV strains) and are HRP-modified can be dry-stored on an interior surface of the receptacle (e.g., on the vial wall as depicted in FIG. 1). Suitable methods for such attachment include those described elsewhere (Sankaranarayanan et al., supra; Stevens et al., Lab on a Chip 8(12):2038-2045, 2008; Ivanova and Kuzmina, Mol Ecol Resources 13(5):890-898, 2013; and Ramachandran et al., Analyst 139(6):1456-1462, 2014). One or more second nucleic acid (e.g., RNA) sequences that are complementary to different nucleic acid sequences from the one or more HPV strains can be immobilized on another interior surface of the receptacle (e.g., on the vial bottom via agarose, as depicted in FIG. 1; Afanassiev et al., Nucl Acids Res 28(12):E66, 2000).

The nucleic acids attached within the receptacle typically have a length sufficient to allow for specific hybridization (e.g., 10-100 nucleotides, 15-75 nucleotides, or 20-50 nucleotides). Nucleic acid sequences for numerous HPV strains include those known in the art. Sequences that are conserved between high-risk HPV strains but are not found within low-risk HPV strains can be particularly useful. Examples of high- and low-risk HPV strains and their nucleotide sequences include the following:

HPV 16 (high-risk):
5′-GCACAGGGACATAATAATGGCATTTGTTGGGGTA
ACCAACTATTTGTTACTGTTGTTGATACTACACGCAGTACAAATATGTCATTATG
TGCTGCCATATCTACTTCAGAAACTACATATAAAAATACTAACTTTAAGGAGTA
CCTACGACATGGGGAGGAATATGATTTACAGTTTATTTTTCAACTGTGCAAAAT
AACCTTAACTGCAGACGTTATGACATACATACATTCTATGAATTCCACTATTTTG
GAGGACTGGAATTTTGGTCTACAACCTCCCCCAGGAGGCACACTAGAAGATAC
TTATAGGTTTGTAACCCAGGCAATTGCTTGTCAAAAACATACACCTCCAGCACC
TAAAGAAGATGATCCCCTTAAAAAATACACTTTTTGGGAAGTAAATTTAAAGG
AAAAGTTTTCTGCAGACCTAGATCAGTTTCCTTTTGGACG-3′ (SEQ ID NO: 1;
GENBANK ® Accession No. S71514.1)
HPV 18 (high-risk):
5′-ATGGTATCCCACCGTGCCGCACGACGCAAACGGG
CTTCGGTAACTGACTTATATAAAACATGTAAACAATCTGGTACATGTCCACCTG
ATGTTGTTCCTAAGGTGGAGGGCACCACGTTAGCAGATAAAATATTGCAATGG
TCAAGCCTTGGTATATTTTTGGGTGGACTTGGCATAGGTACTGGCAGTGGTACA
GGGGGTCGTACAGGGTACATTCCATTGGGTGGGCGTTCCAATACAGTGGTGGA
TGTTGGTCCTACACGTCCCCCAGTGGTTATTGAACCTGTGGGCCCCACAGACC
CATCTATTGTTACATTAATAGAGGACTCCAGTGTGGTTACATCAGGTGCACCTA
GGCCTACGTTTACTGGCACGTCTGGGTTTGATATAACATCTGCGGGTACAACTA
CACCTGCGGTTTTGGATCCCACACCTTCGTCTACCTCTGTGTCTATTTCCACAA
CCACCTTTACCAATCCTGCATTTTCTGATCCGTCCATTATTGAAGTTCCACAAA
CTGGGGAGGTGGCAGGTCCTGTATTTGTTGGTACCCCTACATCTGGAACACAT
GGGTATGAGGAAATACCCCTACAAACATCCGCTTCTTCTGGTACGGGGGAGGA
ACCCATTAGTAGTACCCCATTGCCTACTGTGCGGCGTGTAGCAGGTCCCCGCCT
TTACAGTAGGGCCTACCAACAAGTGTCAGTGGCTAACCCTGAGTTTCTCCCAC
CTCCATCCTCCCCCATTACATATGACAACCCGGCCTTTGAGCCTGTGGACACTA
CATTAACATTTGATCCTCGTAGTGATGTTCCTGATTCAGATTTTATGGATATTATC
CGTCTACATAGGCCTGCTCCAACATCCACCCGTGGGACTGTTCGCTTTAGTAGA
TTAGGTCAACGGGCAACTATGTTTACCCGCAGCGGTACACACCCCGGTGCTAG
GGTTCACTTTTATCATGATATAAGTCCTATTGCACCTTCCCCAGAATATCCTGAA
CTGCAGCCTTTAGTATCTGCCACGGAGGACAATGACTTGTTTGATATATATGCA
GATGACATGGACCCTGCAGTGCCTGTACCATCGCGTTCTACTACCTCCTTTGCA
TCCTTTAAATATTCGCCCACTATATCTTCTGCCTCTTCCTATAGTAATGTAACGGT
CCCTTTAACCTCCTCTTGGGATGTGCCTGTATACACGGGTCCTGATATTACATTA
CCATCTACTACCTCTGTATGGCCCATTGTATCACCCACGGCCCCTGCCTCTACAC
AGTATATTGGTATACATGGTACACATTATTATTTGTGGCCATTACCTTATTTTATTC
CTAAGAAACGTAAACGTGTTCCCTATTTTTTTGCAGATGGCTTTGTGGCGGCC-
3′ (SEQ ID NO: 2; GENBANK ® Accession No. KY502186.1)
HPV 31 (high-risk):
5′-TTATGTATGCGTGTGTACTTGTATATATGTATAGTAT
GTTATGTGTGTATGTATGCTATG61TATGTTAATAAATATGTGTATACCTGTGTGTG
TTGTGTATGTTGTCCTTATATACACCCTTACTATTACTATTTTATAAACTATTGTTC
CTACTTGTTCCTGCTCCTCCCAATAGTCATGTACTTATTTCTGCCTATAATTTAGG
TGTCACGCCATAGTAAAAGTTGTACACCCGGTCCGTTTTTTGCAACTAAAGCTA
CTCCATTTTGATTTTATGCAGCCATTTTAAATCCCTAACCGTTTTCGGTTGCATT
GTTTAAACATGCTAGTACAACTATGCTGATGCAGTAGTTCTGCGGTTTTTGGTT
TCCTGAATACTAGTTTTTGCCAACATTCTGGCTTGTAGTTTCCTGCCTAACACA
CCTTGCCAACATATAATCCAGTCCAACTTTGCAATTATACTATGAATCATGTTTG
TTTAAATACAACTGTAGTTCAACTATGTGTCATGCACATATATTATATTATCCTAC
ACACCTTAAACTGCTTTTAGGCACATATTTTGTAGATTATCTATATCCTTGATTGC
AGTGCTGGCTTTTGCACATGTTTAAACTGCCAAGGTTGTGTCATGCATTATAAA
TAAGTTGTATGTTACTCATATAATTAATTGCATATAGGTATTACACCGTTTTCGGT
TACAGTTTTACAAGCAATTGTTCTTTTTATACTTAATAATAATAATCTTAGTATAA
AAAAGTAGGGAGTGACCGAAAGTGGTGAACCGAAAACGGTTGGTATATAAAG
CACATAGTATTTTGTGCAAACCTACAGACGCCATGTTCAAAAATC-3′ (SEQ ID
NO: 3; GENBANK ® Accession No. JQ693766.1)
HPV 33 (high-risk):
5′-ATGTTTCAGGACACTGAGGAAAAACCACGAACAT
TGCATGATTTGTGCCAAGCATTGGAGACAACTATACACAACATTGAACTACAG
TGCGTGGAATGCAAAAACCCTTTGCAACGATCTGAGGTATATGATTTTGCATTT
GCAGATTTAACAGTTGTATATAGAGAGGGAAATCCATTTGGAATATGTAAACTG
TGTTTGCGGTTCTTATCTAAAATTAGTGAATATAGACATTATAATTATTCTGTATA
TGGACATACATTAGAACAAACAGTTAAAAAACCTTTAAATGAAATATTAATTAG
GTGTATTATATGTCAAAGACCTTTGTGTCCTCAAGAAAAAAAACGACATGTGG
ATTTAAACAAACGTTTTCATAATATTTCGGGTCGTTGGGCAGGGCGCTGTGCGG
CGTGTTGGAGGTCCCGACGTAGAGAAACTGCACTGTGACGTGTAAAAACGCC
ATGAGAGGACACAAGCCAACGTTAAAGGAATATGTTTTAGATTTATATCCTGAA
CCAACTGACCTATACTGCTATGAGCAATTAAGTGACAGCTCAGATGAGGATGA
AGGCTTGGACCGGCCAGATGGACAAGCACAACCAGCCACAGCTGATTACTAC
ATTGTGACCTGTTGTCACACTTGTAATACCACAGTTCGTTTATGTGTCAACAGT
ACAGCAAGTGACCTACGAACCATACAGCAACTACTTATGGGCACAGTGAATAT
TGTGTGCCCTACCTGTGCACAACTATAA-3′ (SEQ ID NO: 4; GENBANK ®
Accession No. KF536963.1)
HPV 39 (high-risk):
5′-GGTACANNNTGTTCT-3′ (SEQ ID NO: 5; GENBANK ®
Accession No. A26661.1)
HPV 45 (high-risk):
5′-GAATTCCAGGCCTAATTTGAGATGTGAGTTGTATC
TGTAACCCAGTGCCCTTGAAGGTGAGGGCAGGCACTCAGCAGCCTCTCCAGG
AAGGCTCACATCCTGGGAGGACTCACTGATTAGTTCTATTGTGTTCATTTGTCT
GTGTCTTAAGCTGAAGGGAAGAGTTAAAACCAAGCCTTTCCCTGGGGGTCTG
GATGAACAGAACTCAACCCAAAGAGTGGCATTGCCTTGTCCTTGGAGCAGGG
AGCTGGGACCCCCCTTGGACTTTGAAAACCAGTGTTTTCAGAATGCAGGTGG
ATAACAAGCCTAAATTTACTTCTGGGCTGAGGAGAGATCTTTGAGGCTCCTGG
AAGGAAACTTGGTGATAAGCCTCCAGTTTGAAACGGCTCTGTCCCTTTAATGT
CTGTGCCTTGACAGCTTTTGGTGAGGAAGCACTTCCTTCCAACAGCTGTCTTC
TTGGCAGAAAACCAAAACATTGGCTTAAAGGGACCCACAGACTGGAACAGCC
TCACATTTCGGCTTTAGAACAAATCCCACAATTGTTCAGCTTTCCGGTCCCCTT
CAGATCAAGCAGAAGATATGTTTTGATTTTCATGCTTGTATTTTAAACAATAATT
TTCTACCCCAGCGTGGTAGTCAATGAGGAGAGAGGGGAAGAATGCGCACATG
ATGCTACACGTTTCTGTTGTTGCTGTTATTATTGGTGGCTTTGAGGAGAGCTGC
TCCCATTTGGGGTTTATACCAACTGTGGATTATGGCTTTGTCATTAAGATTTGAT
CTTTGTTAAATGAAAAACTGTTTATTGTATAAAACTCAGGTTTGTGGACGAAAA
GTTGTTTTTTTTCTTCAGTTAATTAAATTGTTCCTCAAGTTTGTTTAAGGACTTA
AAATCAAACACAACCATGTGTAAACTGCTAAATGAGGCTCCTAAAATGAGAGG
CCTCAACTCTTTAAGTGTGGAGCTAGAAATGTAAATAAGTCCACAGGGCAGAC
TGGTGATTATGATAAAAGCTACCATTTACTGAGCATCTGTCTACTAGGCTCAGC
TCTATGCTAAGTCTACATGTTATCTGTCAAAGTGGTATCATCCCCATTTAATAGC
TGAGGAAACAGAGGCTTAGAAAGGCTGGGTAACTTGACCAGGGTCATGCAAC
TAGTCTGCGGTGGAGCCAGGATTCTGTCTGACCCTAAAGGCCAAGTTCTTTAT
ATTTATTTCTACCACCTGCTAAAGTCTTGAATGGAGGCTGAAAGCACAGTTGG
GGTATGGGGAAGAAAAATATATATACATACATATATGTATATGTATGTATGTATGT
ATGGGGGGTTGTTTTGTTTTTGTTTTTGATAAGGAGTTTTGCTCTTGTTGCCCA
GGCTGGAGTGCAGTGGTATGATCTGGGCTCACTGCAACCTCCGCCTCCCGGGT
TCAAGTCATTCTCCTGCCTCAGCCTCCCGAGTAGCTGGGATTACCGGAGCATG
CCACCACACCCAGCAAAGTTTTGTATTTTTAGTAGAGACAGGGTTTCACCATG
TTGGCCAGGCTGATCTTGAACTCCTCATCTCAGGTGATCTGCCCGCCTCCGCTT
CCCAAAGTGCTGGGATTACAGGTGTGAGTCACCGCGTCCGGCCTACAGATATA
TTTAATTTAAAGAGATCTAAAACAAATACAAAACTGTCCACATCTATGTTGATG
GACCCATAAAAATAGCAGTCTGCCAGGGTCTGCCGGAAGAGACAGATAAGCA
TACATATTAACATGGATATATATGTGAATTTCATTCAAATGGTTCTCACATGAGA
GTAACTAGCATCTTTCTCTCAGATGATGAAGATGATGAAGAGGAAGATGAAGA
GGAAGAAATCGACGTGGTCACTGTGGAGAAGCGGCGTTCCTCCTCCAACACC
AAGGCTGTCACCACATTCACCATCACTGTGCGTCCCAAGAACGCAGCCCTGG
GTCCCGGGAGGGCTCAGTCCAGCGAGCTGATCCTCAAACGATGCCTTCCCATC
CACCAGCAGCACAACTATGCCGCCCCCTCTCCCTACGTGGAGAGTGAGGATGC
ACCCCCACAGAAGAAGATAAAGAGCGAGGCGTCCCCACGTCCGCTCAAGAGT
GTCATCCCCCCAAAGGCTAAGAGCTTGAGCCCCCGAAACTCTGACTCGGAGG
ACAGTGAGCGTCGCAGAAACCACAACATCCTGGAGCGCCAGCGCCGCAACG
ACCTTCGGTCCAGCTTTCTCACGCTCAGGGACCACGTGCCGGAGTTGGTAAAG
AATGAGAAGGCCGCCAAGGTGGTCATTTTGAAAAAGGCCACTGAGTATGTCC
ACTCCCTCCAGGCCGAGGAGCACCAGCTTTTGCTGGAAAAGGAAAAATTGCA
GGCAAGACAGCAGCAGTTGCTAAAGAAAATTGAACACGCTCGGACTTGCTAG
ACGCTTCTCAAAACTGGACAGTCACTGCCACTTTGCACATTTTGATTTTTTTTT
TAAACAAACATTGTGTTGACATTAAGAATGTTGGTTTACTTTCAAATCGGTCCC
CTGTCGAGTTCGGCTCTGGGTGGGCAGTAGGACCACCAGTGTGGGGTTCTGCT
GGGACCTTGGAGAGCCTGCATCCCAGGATGCTGGGTGGCCCTGCAGCCTCCTC
CACCTCACCTCCATGACAGCGCTAAACGTTGGTGACGGTTGGGAGCCTCTGGG
GCTGTTGAAGTCACCTTGTGTGTTCCAAGTTTCCAAACAACAGAAAGTCATTC
CTTCTTTTTAAAATGGTGCTTAAGTTCCAGCAGATGCCACATAAGGGGTTTGCC
ATTTGATACCCCTGGGGAACATTTCTGTAAATACCATTGACACATCCGCCTTTT
GTATACATCCTGGGTAATGAGAGGTGGCTTTTGCGGCCAGTATTAGACTGGAA
GTTCATACCTAAGTACTGTAATAATACCTCAATGTTTGAGGAGCATGTTTTGTAT
ACAAATATATTGTTAATCTCTGTTATGTACTGTACTAATTCTTACACTGCCTGTAT
ACTTTAGTATGACGCTGATACATAACTAAATTTGATACTTATATTTTCGTATGAAA
ATGAGTTGTGAAAGTTTTGAGTAGATATTACTTTATCACTTTTTGAACTAAGAA
ACTTTTGTAAAGAAATTTACTATATATATATGCCTTTTTCCTAGCCTGTTTCTTCC
TGTTCATGTATTTGTTCATGTTTGGTGCATAGAACTGGGTAAATGCAAAGTTCT
GTGTTTAATTTCTTCAAAATGTATATATTTAGTGCTGCATCTTATAGCACTTTGAA
ATAGATTTAGTAGATTGGGTCAAAGGGCAACCATGTTTACACGTAGTGGTAAA
CAAATAGGGGGTAGGGTACATTTTTACCATGATATAAGCCCCATTGCTGCTACA
GAGGAAATTGAATTGCAGCCTTTACTTAGTGCTACAGATGATAGTGACCTGTTT
GATGTATATGCAGACTTTCCACCTCCTGCGTCCACTACACCTAGCACTATAAAC
AAATCATTTACATATCCAAAGTATTCATTGACCATGCCTTCCACTGCTGCATCCT
CTTACAGTAATGTTACAGTACCATTAACATCTGCATGGGATGTACCTATATATAC
TGGCCCGGACATTATATTGCCATCCCATACTCCTATGTGGCCTAGTACATCTCCT
ACCAATGCTGCCACCTCCACCTATATAGGTATTCATGGCACACAATATTATTTAT
GGCCATGGTATTATTATTTTCCTAAAAAACGTAAACGTATTCCCTATTTTTTTGC
AGATGGCTTTGTGGCGGCCTAGTGACAGTACGGTATATCTTCCACCACCTTCTG
TGGCCAGAGTTGTCAACACTGATGATTATGTGTCTCGCACAAGCATATTTTACC
ATGCAGGCAGTTCCCGATTATTAACTGTAGGCAATCCATATTTTAGGGTTGTACC
TAGTGGTGCAGGTAATAAACAGGCTGTTCCTAAGGTATCCGCATATCAGTATAG
GGTGTTTAGAGTAGCTTTGCCCGATCCTAATAAATTTGGATTACCTGATTCTACT
ATATATAATCCTGAAACACAACGTTTGGTTTGGGCATGTGTAGGTATGGAAATT
GGTCGTGGGCAGCCTTTAGGTATTGGCCTAAGTGGCCATCCATTTTATAATAAA
TTGGATGATACAGAAAGTGCTCATGCAGCTACAGCTGTTATTACGCAGGATGTT
AGGGATAATGTGTCAGTTGATTATAAGCAAACACAGCTGTGTATTTTAGGTTGT
GTACCTGCTATTGGTGAGCACTGGGCCAAGGGCACACTTTGTAAACCTGCACA
ATTGCAACCTGGTGACTGTCCTCCTTTGGAACTTAAAAACACCATTATTGAGG
ATGGTGATATGGTGGATACAGGTTATGGGGCAATGGATTTTAGTACATTGCAGG
ATACAAAGTGCGAGGTTCCATTAGACATTTGTCAATCCATCTGTAAATATCCAG
ATTATTTGCAAATGTCTGCTGATCCCTATGGGGATTCTATGTTTTTTTGCCTACG
CCGTGAACAACTGTTTGCAAGACATTTTTGGAATAGGGCAGGTGTTATGGGTG
ACACAGTACCTACAGACCTATATATTAAAGGCACTAGCGCTAATATGCGTGAAA
CCCCTGGCAGTTGTGTGTATTCCCCTTCTCCCAGTGGCTCTATTACTACTTCTGA
TTCTCAATTATTTAATAAGCCATATTGGTTACATAAGGCCCAGGGCCATAACAAT
GGTATTTGTTGGCATAATCAGTTGTTTGTTACTGTAGTGGACACTACCCGCAGT
ACTAATTTAACATTATGTGCCTCTACACAAAATCCTGTGCCAAATACATATGATC
CTACTAAGTTTAAGCACTATAGTAGACATGTGGAGGAATATGATTTACAGTTTAT
TTTTCAGTTGTGCACTATTACTTTAACTGCAGAGGTTATGTCATATATCCATAGT
ATGAATAGTAGTATATTGGAAAATTGGAATTTTGGTGTACCTCCACCACCTACTA
CAAGTTTAGTGGATACATATCGTTTTGTGCAATCAGTTGCTGTTACCTGTCAAA
AGGATACTACACCTCCAGAAAAGCAGGATCCATATGATAAATTAAAGTTTTGGA
CTGTTGACCTAAAGGAAAAATTTTCCTCCGATTTGGATCAATATCCCCTTGGTC
GAAAGTTTTTAGTTCAGGCTGGGTTACGTCGTAGGCCTACCATAGGACCTCGT
AAGCGTCCTGCTGCTTCCACGTCTACTGCATCTAGGCCTGCCAAACGTGTACG
TATACGTAGTAAAAAATAATATGTTAGCACATATATGTATGTTTGTATGTATGGTT
TTGTATGTTGTATGTATGTATGTATTTGTGTGATATATTACTGTATTTTGTTTGTTT
GCGTGCGTGTATGTATGAATGTGCCTTGTGGCATGTATGGTGTTACTGTACATAA
TTGTGGTATTAAATAAAGTATGATACTAGTGTTGTGTAGGGTTGCACCCTTGTG
AGTAACACTACTATTTGTGTGTATGTGTATTGCTTTATACCCTATATTCCTTCCTG
TATTTCAAGTTATAAACTTGCATACTACACAGCATCCATTTTACTTATAATCCTCC
ATTTTGCTGTGCAACCGATTTCGGTTGCCTGTGGCTTATGTTTGACCTTTTAAA
CATAATACTTAAACTGGCACATTTACAACCCCTACATAGTTTAAACTACTGGCG
CGCCTTCTTGGCGTACATGTGGCACACCTGGTATTAGTCATTTTCCTGTCCAGG
TGCACTAAAACAATGGCTTGCACAACTGTATCCACACCCTATGTAATAAAACTG
CTTTTAGGCACATATTTTAGTCTGTTTTTACCTGTGCTAATTGTATAATTGGCATG
TAGAACCACTTTCTTATCCAACAATCTGTCTACTTGTTACATGAACTATAAACTG
ACTCACTTATACATACATAGTTTATGCAACCGAAAAAGGTTGGGCCCTATAACA
CATACCTTTTCTTAATACTTTTAACAATGATACTACATAAAAAAGGGTGTAACCG
AAAACGGTTGCAACCAAAAACGGTGCATATAAAAACTTTGTGGAAAAGTGCA
TTACAGGATGGCGCGCTTTGACGATCCAACGCAACGACCCTACAAGCTACCAG
ATCTGTGCACAGAATTGAATACATCACTACAAGACGTATCTATTGCCTGTGTATA
TTGCAAAGCAACATTGGAACGCACAGAGGTATATCAATTTGCTTTTAAAGATTT
ATTTATAGTGTATAGAGACTGTATAGCATATGCTGCATGCCATAAATGTATAGAC
TTTTATTCCAGAATTAGAGAATTAAGATATTATTCAAACTCTGTATATGGAGAGA
CACTGGAAAAAATAACTAATACAGAGTTGTATAATTTGTTAATAAGGTGCCTGC
GGTGCCAGAAACCATTGAACCCAGCAGAAAAACGTAGACACCTTAAGGACAA
ACGAAGATTCCACAGCATAGCTGGACAGTACCGAGGGCAGTGTAATACATGTT
GTGACCAGGCACGGCAAGAAAGACTTCGCAGACGTAGGGAAACACAAGTATA
GCAATAAGTATGCATGGACCCCGAGCAACACTGCAAGAAATTGTATTGCATTT
GGAACCTCAGAATGAATTAGATCCTGTTGACCTGTTGTGTTACGAGCAATTAA
GCGAGTCAGAGGAGGAAAACGATGAAGCAGATGGCGTTAGTCATGCACAACT
ACCAGCCCGACGAGCCGAACCACAGCGTCACAAAATTTTGTGTGTATGTTGTA
AGTGTGACGGCAGAATTGAGCTTACAGTAGAGAGCTCGGCAGATGACCTTAG
AACACTACAGCAGCTGTTTTTGAGCACCTTGTCCTTTGTGTGTCCGTGGTGTG
CAACTAACCAATAATCTACAATGGCGGATCCAGAAGGTACCGACGGGGAGGG
AACGGGGTGTAATGGCTGGTTTTTTGTAGAAACAATTGTAGAGAAAAAAACA
GGGGATGTAATATCAGATGATGAGGATGAAACCGCAACAGATACAGGGTCGGA
TATGGTAGATTTTATTGACACACAATTATCCATTTGTGAACAGGCAGAGCAAGA
GACAGCACAGGCATTGTTCGATGCGCAGGAAGTTCAGAATGATGCACAGGTG
TTGGATCTTTTAAAACGAAACTTTGCAGGAGGCAGCAAGGAAAACAGTCCAT
TAGGGGAGCTACTCTGGAGGCTGAGGCATGCGAATCTCTTGAACCTGGGAGG
CGGAGGTTGCAGTGAGCCAGTATGGTGCCACTGCACTCCAGCCTGGGCGACA
GAGTGACTCTGTCTCAAAAAAAAGAAAAAGAAAAGAAATCAGCCAGGCATA
GTCCCAGCTACCAGGGAGGTTGAGGCAAGAGAATTGCTTGAACCCAGGAGGC
GGAGGTTGCGGTGAGCTGAGACTCTGTCTCAAAACACACACATACACACATA
CATACACACACACACACACACACACACACACACCCTGAATGGGTGGGTTTAA
ATGAAGATTAAATCCAAGAGGGAAAATAGGTAAATTGGAATATCAAGAAATGA
TCTAGAGTGCAGCACAGAAACAAAGAAAGAAAGGCTAAAAGATGTGAAAAA
GTGGGTGAGAAGGACTAAAACATGCTCAGACTGGAGTTCCGTTAGGACAGGA
GTGAGAAAATGCCCTAACTGACGCCACAGGAGCAGCAGAGACGCATCCTCAC
TGGACCTCACCCAAATCGCAAATTCATGAGCAAAACAAATCATTGTTGTTTTA
GGCCACTAAGTTTAGGTAGTTTTTTATGAAGCAGTAGATAACTGGAATAAGTTG
TGATACCAGGAGTGTGATGTCGCCATATTAAAACACACACACACACACACTCA
CACACATACAACCTAAAAATGTAACATTGGCTTTGGGACCAGATGGTGAGTGG
AAGCCAAAAGGGCTTGCAGAGGCTGCTGGTGAGGACTTAAAAGAAATGCAA
GAGAGGCTTATTGGAATCTAGA-3′ (SEQ ID NO: 6; GENBANK ® Accession
No. AJ242956.1)
HPV 51 (high-risk):
5′-AACAATTATCTTGTAAAAACTAGGGTGTAACCGA
AAAGGGTTATGACCGAAAACGGTGCATATAAAAGTGAAGAGGTAAAAGTATA
GAAGAACACCATGTTCGAAGACAAGAGGGAAAGACCACGAACGCTGCATGA
ATTATGTGAAGCTTTGAACGTTTCTATGCACAATATACAGGTAGTGTGTGTGTAT
TGTAAAAAGGAATTATGTAGAGCAGATGTATATAATGTAGCATTTACTGAAATTA
AGATTGTATATAGGGATAATAATCCATATGCAGTATGCAAACAATGTTTACTGTT
TTATTCAAAAATTAGAGAGTATAGACGTTATAGCAGGTCTGTGTATGGTACTAC
ATTAGAGGCAATTACTAAAAAAAGCTTATATGATTTATCGATAAGGTGTCATAG
ATGTCAAAGACCACTTGGGCCTGAAGAAAAGCAAAAATTGGTGGACGAAAAA
AAAAGGTTCCATGAAATAGCGGGACGTTGGACGGGGCAATGCGCTAATTGCTG
GCAACGTACACGACAACGTAACGAAACCCAAGTGTAATAAAGCCATGCGTGG
TAATGTACCACAATTAAAAGATGTAGTATTGCATTTAACACCACAGACTGAAAT
TGACTTGCAATGCTACGAGCAATTTGACAGCTCAGAGGAGGAGGATGAAGTA
GATAATATGCGTGACCAGCTACCAGAAAGACGGGCTGGACAGGCTACGTGTTA
CAGAATTGAAGCTCCGTGTTGCAGGTGTTCAAGTGTAGTACAACTGGCAGTGG
AAAGCAGTGGAGACACTCTTCGCGTTGTACAGCAGATGTTAATGGGCGAACT
GAGCCTGGTTTGCCCGTGTTGTGCGAACAACTAGCAACGGCGATGGACTGTG
AAGGTACAGAGGATGAGGGGGCGGGGTGTAATGGGTGGTTTTTTGTTGAAGC
AATAGTAGAAAAAAAAACAGGAGATACTGTTTCGGATGATGAGGATGAAAATG
CAGATGATACAGGATCTGATTTAATAAACTTTATAGATAGTGAAACTAGTATTTG
CAGTCAGGCGGAACAGGAGACAGCACGGGCGTTGTTTCAGGCCCAAGAATTA
CAGGCAAACAAAGAGGCTGTGCATCAGTTAAAACGAAAGTTTCTAGTCAGCC
CGCGAAGCAGCCCATTAGGAGACATTACAAATCAAAACAACACACACAGCCA
TAGTCAGGCAAACGAGTCACAAGTTAAAAGGAGATTACTGGACAGTTATCCG
GACAGCGGATATGGCAATACACAAGTGGAAACTGTGGAAGCAACGTTGCAGG
TAGATGGGCAACATGGCGGTTCACAGAACAGTGTGTGTAGTAGCGGGGGGGG
CAGTGTTATGGATGTGGAAACAACAGAAAGCTGTGAAAATGTAGAACTAAAC
AGTATATGTGAAGTATTAAAAAGCAGTAATGCAAAAGCAACGTTAATGGCAAA
ATTCAAAGAGTTGTATGGTATTAGTTATAATGAGTTGGTACGGGTGTTTAAAAG
TGATAAAACATGTTGTATAGATTGGGTTTGTGCATTGTTTGGCGTTTCCCCAATG
GTAGCAGAAAATTTAAAAACACTAATTAAGCCATTTTGCATGTACTACCATATA
CAATGTTTATCATGTGATTGGGGCACCATTGTATTAATGCTAATTAGGTTTTCAT
GTGCAAAAAACAGAACAACAATTGCTAAGTGTTTAAGTACATTAGTAAATATC
CCACAATCACAAATGTTTATAGAACCACCAAAATTACGTAGTACACCTGTGGC
ATTATATTTTTATAGAACAGGCATATCAAACATTAGCAATACATATGGAGAGACA
CCTGAATGGATTACACGACAAACGCAACTACAACATAGTTTTGAGGATAGTAC
CTTTGAATTATCACAAATGGTGCAATGGGCATTTGACCATGAAGTATTAGATGA
TAGTGAAATAGCATTTCATTATGCACAATTAGCAGATATAGATAGTAATGCTGCA
GCGTTTTTAAAGAGTAATTGCCAAGCAAAATATGTAAAAGATTGTGGGACCAT
GGCACGGCATTACAAACGAGCACAAAGAAAATCATTATCCATGTCAGCCTGGA
TAAGGTATAGATGTGATAGAGCAAAGGATGGAGGCAACTGGAGAGAAATTGCT
AAATTTTTAAGATATCAAGGTGTAAACTTTATGTCCTTTATTCAAATGTTTAAAC
AGTTTTTAAAAGGAACACCAAAACACAATTGCATAGTTATATATGGCCCACCAA
ACACAGGCAAGTCATTATTTGCAATGAGCCTAATGAAGTTTATGCAAGGGTCC
ATTATTTCATATGTAAACTCTGGTAGTCATTTTTGGTTACAGCCACTAGAGGATG
CTAAAATAGCATTGTTAGATGATGCTACGTATGGGTGTTGGACATATATTGATCA
GTATTTAAGAAACTTTTTAGATGGTAATCCATGTAGTATAGATAGAAAACATAGG
AGTTTAATACAATTAGTATGTCCACCATTACTAATAACGTCAAACATAAATCCAC
AAGAGGATGCAAACCTAATGTATTTACATACAAGGGTAACAGTATTAAAGTTTT
TAAATACATTTCCATTTGATAACAATGGGAATGCTGTGTATACATTGAATGATGA
AAATTGGAAAAATTTTTTTTCCACCACATGGTCCAGATTAGATTTGGAGGAGG
AAGAGGACAAAGAAAATGGAGACCCTATGCCACCGTTTAAATGTGTGCCAGG
AGAAAATACTAGACTGTTATGAACTGGACAGTGATAAATTAGTAGATCAAATTA
ACTATTGGACATTGTTACGATATGAAGCTGCTATGTTTTATGCAGCACGGGAAA
GAAACTTACAAACAATCAATCACCAGGTAGTACCAGCAACAACAGTATCAAA
ACAAAAGGCCTGTCAAGCAATTGAAATGCACATGGCCTTACAATCGCTTAACA
AATCAGACTATAACATGGAACCATGGACAATGCGGGAGACATGTTATGAACTAT
GGTGTGTGGCTCCCAAGCAATGTTTCAAAAAGGGGGGCATAACTGTAACAGTT
ATATTTGATGGAAATAAGGACAATGCAATGGACTATACAAGCTGGAAATTTGTA
TATATATATGATAATGATAAGTGGGTAAAGACAGATGGAAATGTGGACTATACG
GGTATATATTACACTGTAAATTCAAAAAAAGAGTATTATGTACAGTTTAAAGATG
AAGCCAAAAGATATGGGGCACAACAGTGGGAGGTCTATATGTATGGTACTGTA
ATAACATGTCCTGAATATGTATCTAGTACCTGCAGCGACGCGTTATCCACTACTA
CAACTGTTGAACAACTATCAAACACCCCAACGACCAATCCCCTTACCACCTGC
GTGGGCGCCAAAGAAGCCCAGACACAACAGCGAAAACGACAGCGACTTACT
GAGCCCGACTCCTCCACAATCTCCCCACTGTCCGTGGACAATACAGACAACCA
AATACACTGTGGAAGTGGAAGCACTAACACTGGAGGGCACCAAAGTGCAACT
CAGACTGCGTTTATAGTGCATTTAAAAGGTGATACAAATTGTTTAAAATGTTTT
AGATACAGATTTACAAAACACAAAGGGTTATATAAAAACGTATCCTCAACCTG
GCATTGGACCAGTAATACTAAAACAGGCATTGTTACCATTGTGTTTGACAGTGC
ACATCAACGGGAAACATTTATAAAAACCATTAAAGTACCCCCAAGTGTAACAC
TGTCATTGGGAATTATGACACTGTAACTACTGTAATATATGTATTGTACATATATA
CTGTCACAAGCCAATATGTGCTGCTAAGTGTATAGACATATAGTAACCATTGCA
GTGTTTATTATTTTGCTATTTGTGCTTTGCGTGTGTGTGTGTCTTGTGTTGTGTT
GTTTGTTGCCGCTACTGCTGTCCCAATACGTGTTTGCAGCTGCCTTATTATTAAT
TTTATGTTTTTGGTTTGTTGTTGCAACATCCCAATTAACTACATTTTTTGTATATT
TGATTTTTTTTTACTTACCTTGTTTACTTTTACATCTATATACATTTTTACTTTTGC
AATAAACTTGTTATATTTTTGTGATTAAATATGGTGGCTACACGTGCACGGCGTC
GGAAGCGGGCATCTGTAACACAATTATATTCTACATGCAAAGCTGCTGGTACAT
GTCCTCCTGATGTTGTGAATAAGGTTGAAGGTACTACATTGGCCGATAAAATAT
TACAGTGGAGTGGGTTAGGTATATTTTTGGGTGGCCTAGGTATTGGTACTGGGT
CTGGATCTGGGGGGCGTACTGGATATATCCCTTTAGGTGGTGGGGGTCGCCCA
GGCGTGGTGGATATTGCTCCTGCAAGGCCACCTATTATAATTGAACCTATAGCA
CCTACTGAACCTTCTATAGTAAATTTGGTTGAAGACTCTAGTATTATTCAGTCTG
GGTCTCCTATACCTACCTTTACTGGTACCGATGGCTTTGAAATTACTTCATCTTC
CACAACAACCCCTGCTGTGTTGGACATAACCCCATCTGCTGGTACTGTACATGT
TTCCAGTACTAACATTGAAAATCCTTTATATATTGAACCTCCATCCATTGAGGCT
CCACAATCTGGAGAAGTGTCAGGACATATATTTACTAGTACACTTACTTCTGGT
ACTCATGGGTATGAAGAAATACCTATGGAAGTGTTTGCATCCAATGTCAGTACT
GGTACTGAACCTATTAGCAGCACACCTACTCCAGGGGTTAGTCGCATAGCTGC
TCCCCGCTTGTATAGTAAGTCTTACACACAAGTTAAAGTTACAAATCCTGATTT
TATTAGTAAGCCATCCACATTTGTTACATTTAATAATCCTGCTTTTGAGCCTATTG
ACACATCCATAACTTTTGAGGAACCTGATGCTGTTGCACCTGATCCTGATTTTC
TGGATATTATTAAACTGCACCGCCCTGCCCTTACATCTCGTAGAGGCACAGTAC
GCTTTAGTAGGTTAGGTCAAAAGGCCACCATGCGCACTCGTAGTGGCAAACAA
ATTGGTGCTCGTGTACATTATTATCATGATATTAGTAGAATTGCACCAGCTGATG
AACTTGAAATGCAGCCTTTACTTTCCCCTTCTAATAATTATAGTTATGACATTTAT
GCTGATTTAGATGAAGCTGAAACAGGTTTTATACAGCCCACACACACCACACC
TATGTCACACTCCTCTTTGTCTAGGCAGTTGCCCTCCTTATCTTCATCTATGTCT
TCATCTTATGCAAATGTTACTATTCCATTTTCAACTACATATTCTGTTCCTATTCA
TACAGGGCCTGATGTGGTATTGCCCACATCTCCTACAGTATGGCCTTATGTTCCC
CACACTTCCATTGACACCAAGCATTCTATTGTTATACTAGGTGGGGATTACTATT
TGTGGCCCTATACACATTTACTACGCAAACGCCGTAAACGTATACCCTATTTTTT
TACAGATGGCATTGTGGCGCACTAATGACAGCAAGGTGTATTTGCCACCTGCA
CCTGTGTCTCGAATTGTGAATACAGAAGAATATATCACACGCACCGGCATATAT
TACTATGCAGGCAGTTCCAGACTAATAACATTAGGACATCCCTATTTTCCAATAC
CTAAAACCTCAACGCGTGCTGCTATTCCTAAAGTATCTGCATTTCAATACAGGG
TATTTAGGGTACAGTTACCAGATCCTAACAAGTTTGGACTCCCGGATCCAAATT
TATATAATCCAGACACAGATAGGTTGGTGTGGGGTTGTGTGGGCGTTGAGGTG
GGCAGAGGACAGCCCCTTGGTGTTGGCCTTAGTGGTCATCCCTTATTTAATAAA
TATGATGACACAGAAAATTCACGCATAGCAAATGGCAATGCACAACAAGATGT
TAGAGATAACACATCTGTTGACAACAAACAGACTCAGTTATGTATAATAGGCTG
TGCTCCACCTATTGGGGAACACTGGGGTATTGGCACTACATGCAAAAACACAC
CTGTACCTCCAGGAGACTGCCCCCCCCTGGAACTTGTATCCTCTGTCATTCAGG
ATGGCGATATGATTGATACAGGGTTTGGAGCTATGGATTTCGCTGCCCTACAGG
CCACCAAATCAGACGTCCCTTTGGATATTTCACAGTCTGTTTGTAAATATCCTG
ATTATTTAAAAATGTCTGCAGACACATATGGTAATTCCATGTTTTTTCATTTACG
CAGGGAGCAAATCTTTGCTAGGCACTATTATAATAAACTTGGTAGTGTTGGGGA
AGACATTCCTAACGATTATTATATTAAGGGTAGTGGTAATGGCCGTGACCCTATA
GAAAGTTATATATACTCTGCTACTCCCAGTGGGTCTATGATAACATCTGATTCTC
AGATTTTTAATAAGCCTTATTGGCTCCACCGTGCGCAGGGTCACAATAATGGCA
TTTGCTGGAACAATCAGCTTTTTATTACCTGTGTTGATACTACCAGAAGTACAA
ATTTAACTATTAGCACTGCCACTGCTGCAGTTTCCCCACCATTTACTCCAAGTA
ACTTTAAGCAATATATTAGGCATGGGGAAGAGTATGAATTGCAATTTATTTTTCA
GTTATGTAAAATTACTTTAACTACAGAGGTAATGGCTTATTTACACACAATGGAT
CCTACCATTCTTGAACAGTGGAATTTTGGATTAACATTACCTCCGTCTGCTAGT
TTGGAGGATGCATACAGGTTTGTTAGAAATGCAGCTACTAGCTGTCAAAAGGA
CACCCCTCCACAGGCTAAGCCAGATCCTTTGGCCAAATATAAATTTTGGGATGT
TGATTTAAAGGAACGATTTTCTTTAGATTTAGACCAATTTGCATTGGGTCGCAA
GTTTTTGTTGCAGGTTGGCGTACAACGCAAGCCCAGACCAGGCCTTAAACGCC
CGGCCTCATCGGCATCCTCTTCCTCTTCCTCTTCAGCCAAACGTAAACGTGTTA
AAAAGTAATGTATGTTAGTTTTTGTATGCTTGTGCACACTGTTGTATGCCTGTAT
GTATATGTTTGTGTATGTACTGTATGTGTTTTTGTGTGTGTGTGTGTTGTTGTTCC
TGTATGTATGAGTTATGTATGTTTATTATTAATAAACTATGTGGTGTGTGTGTGTG
TGTTTTTGCATGACTGCATTTGTATGACATGTACGGGTGTATGTGGGTATTACAT
TATCCCCGTAGGTCAAGGGTGGTGTTTCGGTGGCGTCCCTATTGCCCTACCCAT
TTTTTTGCAGCACAACAGTTTATATTTATGCTATTTAGTTATACTTTGTAGCTTCC
ATTTTGTTACAGCTGCAGCCATTTTGAGTGCAACCGATTTCGGTTGCTGTACTT
TTAGTATATTTGCCAAGTTTTAAACCACAACTGCCAGTTGTTTTTGGCATAAAC
CATCAGTTTTTTATGACATAGTGCATACATCCGCCCGCCCACGCCTTGTACTTG
GCGCGCCTTACCGGCGCTAGTCATACAACCTATTAGTCATTTGTACTTTAACAAT
TGTTGGCACACTGTTTTCCGCCCTATAATAATTTAACTGCTTATAGGCATGTATT
TTTTGGCATATTTTATCTTACTAATTGCATAGTTGGCAGGTCAAATACTATGTTTT
TAGTGCCAAGTTTCTATCCTACTTATAAACCATCTTACTCATATGCAGGTGTGCT
ACACAAATGTGTTACCTAACCGATTTGTGTTCTGCCTATGCTTGCAACATTTTTT
CTTATAACATTT-3′ (SEQ ID NO: 7; GENBANK ® Accession No.
KT725857.1)
HPV 6 (low-risk):
5′-GCCCAGGGACATAACAATGGTATTTGTTGGGGTAAT
CAACTGTTTGTTACTGTGGTAGATACCACACGCAGTACCAACATGACATTATGT
GCATCCGTAACTACATCTTCCACATACACCAATTCTGATTATAAAGAGTACATGC
GTCATGTGGAAGAGTATGATTTACAATTTATTTTTCAATTATGTAGCATTACATT
GTCTGCTGAAGTAATGGCCTATATTCACACAATGAATCCCTCTGTTTTGGAAGA
CTGGAACTTTGGGTTATCGCCTCCCCCAAATGGTACATTAGAAGATACCTATAG
GTATGTGCAGTCACAGGCCATTACCTGTCAAAAGCCCACTCCTGAAAAGGAA
AAGCCAGATCCCTATAAGAACCTTAGTTTTTGGGAGGTTAATTTAAAAGAAAA
GTTTTCTAGTGAATTGGATCAGTATCCTTTGGGACG-3′ (SEQ ID NO: 8;
GENBANK ® Accession No. S73503.1)
HPV 11 (low-risk):
5′-ATGTGGCGGCCTAGCGACAGCACAGTATATGTGCC
TCCTCCTAACCCTGTATCCAAAGTTGTTGCCACGGATGCTTATGTTAAACGCAC
CAACATATTTTATCATGCCAGCAGTTCTAGACTTCTTGCAGTGGGTCATCCTTAT
TATTCCATAAAAAAGGTTAACAAAACTGTTGTGCCAAAGGTGTCAGGATATCA
ATACAGAGTATTTAAGGTGGTGTTACCAGATCCTAACAAATTTGCATTGCCTGA
CTCGTCTCTTTTTGATCCCACAACACAACGTTTGGTATGGGCATGCACAGGCCT
AGAGGTGGGCCGGGGACAGCCATTAGGTGTGGGTGTAAGTGGACATCCTTTAC
TAAATAAATATGATGATGTTGAAAATTCAGGGGGTTACGGTGGTAACCCTGGAC
AGGATAACAGGGTTAATGTAGGTATGGATTATAAACAAACACAATTATGCATGG
TTGGATGTGCCCCCCCTTTGGGCGAGCATTGGGGTAAAGGTACACAGTGTAGT
AATACATCTGTACAGAATGGTGACTGCCCGCCCTTAGAACTTATTACCAGTGTT
ATACAGGATGGCGATATGGTTGACACAGGCTTTGGTGCTATGAATTTTGCTGAT
TTGCAGACCAATAAATCAGATGTTCCTCTTGACATATGTGGCACTGTATGTAAA
TATCCAGATTATTTACAAATGGCTGCAGACCCATATGGTGATAGATTATTTTTTTA
TCTACGGAAGGAACAAATGTTTGCCAGACATTTTTTTAACAGGGCTGGTACCG
TGGGGGAACCTGTGCCTGATGATCTTTTAGTTAAGGGTGGTAACAATCGCTCG
TCTGTAGCGAGTAGTATATATGTTCACACCCCAAGCGGCTCTTTGGTGTCCTCT
GAGGCACAATTGTTTAATAAGCCATATTGGCTACAAAAAGCCCAGGGACATAA
CAATGGTATTTGTTGGGGTAATCATCTGTTTGTTACTGTGGTAGATACCACACG
CAGTACCAACATGACATTATGTGCATCCGTATCTAAATCTGCCACATACACCAAT
TCTGATTATAAAGAGTACATGCGTCATGTGGAAGAGTTTGATTTACAATTTATTT
TTCAATTATGTAGCATTACATTGTCTGCTGAAGTAATGGCCTATATTCACACAAT
GAATCCCTCTGTTCTCGAAGACTGGAACTTTGGGTTATCGCCTCCCCCAAATG
GTACACTCGAGGATACCTATAGGTATGTGCAGTCACAGGCCATTACCTGTCAAA
AGCCCACTCCTGAAAAGGAAAAGCAAGATCCCTATAAGGACATGAGTTTTTGG
GAGGTTAATTTAAAAGAAAAGTTTTCTAGTGAATTGGATCAGTTTCCTTTGGGA
CGCAAGTTTTTGTTACAAAGTGGATATAGGGGACGGACCTCTGCTCGTACCGG
TATTAAGCGCCCTGCTGTTTCCAAACCCTCTACTGCCCCTAAACGTAAGCGCAC
CAAAACTAAAAAGTAA-3′ (SEQ ID NO: 9; GENBANK ® Accession No.
U55993.1)
HPV 42(low-risk):
5′-CTTATTATAAACTACAATCCTGGCTTTGAAAAATAA
GGGAGTAACCGAATTCGGTTCAACCGAAACCGGTACATATATAAACCACCCAA
AGTAGTGGTCCCAGTTAAGGCAGAATGTCAGGTACATCTGCCTCATCACAGCC
ACGCACATTATACCAATTGTGTAAGGAATTTGGGCTGACATTGCGGAATTTACA
GATTTCCTGCATTTGGTGCAAAAAGCACTTAACAGGCGCAGAGGTGCTCGCGT
ACCATTTTAAAGATTTGGTAGTGGTGTGGAGGAAGGACTTTCCATATGCTGCAT
GTGCATTTTGTTTAGAATTTAATTCTAAAATTTGTGCACTGCGACACTACGAAA
GATCAGCATTTTGGTATACAGTGGAGAAAGAAACTGGACTACTTTTAGAAGAA
CAACAAATTAGATGTGCCTTGTGTCAAAAGCCGTTATCACAGAGCGAAAAAA
ACCATCATATTGATACAGGTACAAGATTTCAATTTATATTGTGTCAGTGGACGGG
TCGGTGTACGCATTGCAGAGGACAATGCGTGGAGAGACGCCTACCCTAAAGG
ACATTGTTTTGTTTGACATACCAACGTGTGAGACACCCATTGACCTGTATTGCT
ATGAACAATTGGACAGCTCAGATGAAGATGACCAAGCCAAACAGGACATACA
GCGTTACAGAATACTGTGTGTGTGTACACAGTGTTACAAGTCTGTTAAACTCGT
TGTGCAGTGTACAGAGGCGGACATAAGAAACCTGCAACAGATGCTTTTGGGC
ACACTGGATATTGTGTGTCCTTTGTGTGCCCGCGTGGAGTAACTGCAATGGCG
GATGATACAGGTACAGAGGAGGGGCTAGGGTGTTCTGGATGGTTTTGTGTAGA
AGCTATAGTAGACAAAACAACAGAAAATGCTATTTCAGATGACGAGGACGAA
AATGTAGACGATAGTGGGTTAGATCTTGTGGATTTTGTAGATAATAGTACAGTA
ATACATACAAAGCAGGTACATGCACAAGCCTTATTAAATAAACAACAAGCACA
TGCAGATCAGGAGGCAGTACAGGCACTAAAACGAAAGCTATTAGGCAGTCCA
TATGAAAGCCCTGTCAGTGATTCACAGCACAGCATAGACAACGAACTAAGTCC
TAGGCTTGGCGGTTTAACGCTATGTCGGGGGTCCCAAGGGGCCAAACGACGAT
TATTCCAGTCACTGGAAAATCGAGACAGTGGATATGGCTATTCTGAAGTGGAA
GTACAGCAGACACAGGTAGAACACGGACATGGCGCCGTACATGGGACTATGG
GTAACGGGGGGGCAGTGGGTAGTGAACTTGGGGTGCAGGAAAATGAAGAAG
GTAGTACTACAAGTACGCCTACAACAAGGGTGGTAGAATTACTTAAGTGTAAG
AACCTGCATGCAACATTGTTAGGTAAGTTTAAAGAATTGTTTGGAGTGTCATTT
GGCGATTTAGTAAGACAGTTTAAAAGTGACAAAAGCAGTTGTACAGACTGGG
TTATTGCAGCATTTGGGGTTAATCATAGTATTGCAGAAGGGTTTAATACATTAAT
TAAAGCAGATTCACTATATACACATATACAATGGCTAACCTGTACGTGGGGCAT
GGTGTTATTAATGCTAATTAGATTTAAATGTGGAAAAAATCGTACTACAGTGTC
CAAAGGCCTTAGTAAATTATTAAACATACCTACAAATCAATTATTAATAGAGCC
ACCTCGGTTACAAAGTGTGGCTGCCGCCATATACTGGTTTAGATCAGGAATATC
TAATGCTAGCATTGTAACCGGAGACACACCAGAGTGGATTCAAAGACAAACA
ATTTTAGAACATTGTTTTGCAGATGCCCAATTTAATTTAACAGAAATGGTGCAA
TGGGCATATGATAATGATATTACTGAAGACAGTGACATTGCATATGAATATGCAC
AACGGGCAGACAGGGATAGCAATGCTGCTGCATTTTTAAAAAGTAACTGCCAG
GCAAAATATGTAAAAGATTGTGGCGTCATGTGCAGACATTATAAAAAAGCACA
AATGAGACGTATGTCTATGGGTGCATGGATAAAACATAGAAGTGCCAAGATAG
GGGATAGTGGAGATTGGAAACCTATAGTAAAATTTATTAGATATCAACAAATTG
ATTTTTTAGCATTTATGTCTGCATTTAAAAAGTTTTTACATAATATACCTAAAAA
AAGTTGTTTAGTGTTAATTGGTCCTCCAAATACAGGAAAATCACAGTTTGGAAT
GAGTTTAATAAACTTCTTAGCAGGAACTGTAATATCATTTGTAAATTCACATAGC
CATTTTTGGCTGCAGCCATTGGACAGTGCAAAAATAGCTATGCTGGATGATGCA
ACTCCACCATGTTGGACATATTTAGATATATATTTAAGAAATTTATTAGATGGCA
ATCCATGCAGTATAGATAGAAAACATAAAGCATTAACAGTTGTTAAGTGCCCAC
CATTACTTATAACATCAAATACAGATATTAGAACAAATGACAAATGGAAATACC
TATACAGCAGAGTTAGTTTATTTGAATTTCCAAATCCATTTCCATTAGATACAAA
TGGAAATCCTGTATATGAATTAAATGACAAAAATTGGAAATCATTTTTTCAAAG
GTTGTGGTCCAGCTTAGAATTTCAAGAATCAGAGGACGAGGAAGACTATGGA
GAGACTGGCCAAACGTTTAGATGCGTGCCAGGAACAGTTGTTAGAACTGTATG
AGGAAAATAGTAGGGATTTACAAAAACATATTGAACATTGGAAATGTTTACGTA
TGGAGGCAGTGGTATTGTATAAGGCCCGTGAAATGGGCTTTGCAAATATAGGA
CATCAAATAGTACCAACATTGGAAACATGTAGAGCCAAGGCCCACATGGCAAT
TGAAATACACTTGGCATTAGAGACATTATTGCAGTCCTCGTATGGTAAAGAACC
ATGGACATTGCAAGAAACAAGTAATGAACTGTGGCTTACGAATCCTAAAAAAT
GTTTTAAAAAACAAGGACGTACCGTGGAGGTTATATTTGATGGAAAACAGGAC
AATGCAATGCATTATACAGCATGGACATATATATATATACAAACTGTGCAAGGTA
CATGGTGTAAAGTACAAGGACACGTTTGCCATGCAGGACTATATTATATTGTGG
AAAATATGAAACAGTTTTATTGTAATTTTAAAGAGGAGGCAAAAAAATATGGG
GTAACAGACCAATGGGAGGTACATGATGGCAATCAGGTGATTGTTTCTCCTGC
ACCCATATCTAGCACCACATCCACCGACGCAGAGATACCCTCTACTGGATCTAC
TAAGTTGGTACAACAAGTGTGCACCACAAACCCATTGCACACCACAACGTCC
ATTGACAACCACCACGCAGACTGTACAGACGGAACAGCATACAACGTGCCCA
TCCAAACCTCACCGCCACGAAAACGATACAGACAGTGTGGACAGTCGCCATC
ACAGCACCTGCAGCACTCAAACCCCAGCATCCCCAGCATCCCCAGCGCATCCG
TGGACCCTGGATTGTGTGGGGTCAGAACTAACAGTGAAAACTGTAACAAGCG
ACGGAACCACTGTGGAAGTCAGGCTACGCCTGTAATTCATTTACAAGGTGACC
CTAATTGCCTAAAATGCCTACGATTTAGGCTAAAAAGAAATTGTTCACATTTATT
TACACAGGTGTCATCTACATGGCATTTAACAGAAAATGATTGTACACGTGACAC
TAAAACTGGTATAATAACAATACATTATTATGATGAAGCACAAAGAAATTTATTT
TTAAATACTGTAAAAATACCTTCTGGGATAAAATCCTGTATTGGATATATGTCTAT
GTTACAGTTTATATGATTAGTTGTATATGTGTATAAACAGTTATAGGACTTCAATA
CTGTGACTCCACAACGTGTGGGACAACCGGCCAGAAACTGCTGCTTTTATTGT
TTATAGTTGTTGGTGCGTGTGTTGTGTGTGTGTGGATTAGTTTACAAAATTATCC
ATATCCTGTATGGGCCTCTTGCCTTGCTAGCTACCTAACATTGGTGCTATTATCAT
GGTTGCAGGTACTAACATACTTTGACTATTTTTTTCTATGTTTAATCATTCTTGGT
ATTCCTTCTGTCTTACTAACATTACTAATACATTTAGCAATACAATAACACATATT
AGTTTAGGTGTGTGTGTGTGGTGTGCATGTGATTTGTACATGGTTGTACATATAT
AATACCAATTATTGTTTGGCTACTATTTTCATTTATAGCCACACTGCTGTTTTGC
ATATTGGTATTACAAACATATAAACTGTTACCATACGTATATACAGTGCTGTAAA
TAAACTTTTGTTATATTGTGTGTACTTCTTTTGTGCTATTACAATGCCACCACAA
CGGTCCCGCAGACGAAAGCGGGCCTCTGCCACACAATTATATCAAACGTGTAA
GGCCTCAGGGACATGTCCTCCAGATGTTATTCCCAAAGTTGAAGGAACCACAT
TGGCAGATAAAATTTTACAATGGGGTAGTTTAGGCGTGTTTTTTGGGGGGTTGG
GAATTGGCACTGGTGCAGGTACGGGTGGGCGCACGGGCTATGTGCCTCTGGG
AACAAGGCCTCCTGTAATTGCTGAACCAGGACCTGCAGTACGCCCACCAATAG
CTGTTGACACCGTGGGGCCATCTGATCCTTCTATTGTTTCCTTATTAGAAGAGT
CATCAGTTATTGATGCAGGAATAACAGTACCTGATATTACTTCTCATGGAGGTTT
TAATATTACTACATCTACTGGTGGGCCTGCCTCAACGCCTGCTATATTAGATATC
TCCCCTCCCACTAATACTATACGTGTCACAACAACTACATCTACCAATCCTTTAT
ATATTGATCCTTTTACATTGCAGCCGCCATTGCCAGCAGAGGTTAATGGGCGCC
TATTAATATCTACTCCTACCATCACACCCCACTCATATGAAGAAATACCAATGGA
CACGTTTGTTGTATCTACAGATACAACTAACACATTTACTAGTACTCCCATTCCT
GGCCCTCGGTCGTCTGCACGCCTGGGGTTATATTCTAGAGCAACGCAACAACG
TCCAGTTACTACCAGTGCATTTTTAACATCTCCTGCACGGTTGGTTACTTATGAC
AATCCAGCCTATGAAGGACTTACGGAGGATACATTAGTATTTGAACATCCATCC
ATTCATACTGCACCTGACCCTGATTTCATGGATATAGTTGCATTGCATCGTCCTA
TGTTATCATCCAAACAGGGTAGTGTACGTGTTAGTAGAATTGGACAAAGGCTG
TCTATGCAGACACGTCGCGGGACCCGTTTTGGGTCACGTGTACACTTTTTTCAT
GACCTTAGCCCTATTACACACTCTTCAGAAACTATTGAATTACAGCCTTTATCTG
CTTCTTCAGTATCTGCAGCCTCCAATATTAATGATGGGTTATTTGATATTTATGTT
GATACTAGTGATGTAAATGTTACAAATACCACTTCCTCTATACCTATGCATGGTT
TTGCTACCCCCCGTTTGTCCACTACATCTTTCCCTACATTACCTAGCATGTCTAC
ACATTCTGCCAATACCACCATACCTTTTTCGTTTCCTGCCACTGTGCATGTGGG
CCCTGATTTATCTGTTGTGGACCACCCATGGGACAGTACCCCAACGTCTGTAAT
GCCTCAGGGTAACTTTGTAATGGTATCAGGATGGGATTTTATATTGCATCCTAGT
TATTTTTGGCGTAGGCGCCGTAAACCTGTACCATATTTTTTTGCAGATGTCCGTG
TGGCGGCCTAGTGACAACAAGGTTTATCTACCTCCTCCTCCTGTTTCCAAGGTG
GTCAGCACTGATGAATATGTGCAACGCACCAACTACTTTTACCATGCCAGCAG
TTCTAGGCTATTGGTTGTTGGTCACCCTTATTACTCTATTACAAAAAGGCCAAAT
AAGACATCTATCCCCAAAGTGTCTGGTTTACAGTACAGAGTATTTAGAGTTAGG
CTCCCTGATCCTAATAAGTTTACATTGCCTGAAACTAATTTATATAACCCAGAGA
CACAGCGCATGGTGTGGGCCTGTGTGGGGCTAGAAGTAGGTCGTGGACAGCC
TTTGGGCGTTGGTATTAGTGGCCATCCATTATTGAATAAGTTGGATGATACTGAA
AATGCGCCTACATATGGTGGAGGCCCTGGTACAGACAATAGGGAAAATGTTTC
TATGGATTATAAACAAACACAGTTGTGTTTAGTTGGCTGTAAACCTGCCATAGG
GGAGCACTGGGGTAAAGGTACTGCCTGTACACCACAGTCCAATGGTGACTGC
CCACCATTAGAATTAAAAAATAGTTTTATTCAGGATGGGGATATGGTGGATGTA
GGGTTTGGGGCACTAGATTTTGGTGCTTTACAATCCTCCAAAGCTGAGGTACC
TTTGGATATTGTAAATTCAATTACTAAATATCCTGATTACTTAAAAATGTCTGCT
GAGGCCTATGGTGACAGTATGTTTTTCTTTTTAAGGCGAGAACAAATGTTTGTT
CGTCATTTGTTTAATAGGGCTGGCGCAATTGGTGAACCTGTACCTGATGAACTG
TATACCAAGGCTGCTAATAATGCATCTGGCAGACATAATTTAGGTAGTAGTATTT
ATTATCCTACCCCTAGTGGTTCTATGGTAACATCTGATGCACAACTATTTAATAA
ACCATATTGGTTACAACAAGCACAAGGACACAATAATGGTATATGTTGGGGAA
ATCAGCTATTTTTAACTGTGGTTGATACTACCCGTAGTACTAACATGACTTTGTG
TGCCACTGCAACATCTGGTGATACATATACAGCTGCTAATTTTAAGGAATATTTA
AGACATGCTGAAGAATATGATGTGCAATTTATATTTCAATTGTGTAAAATAACAT
TAACTGTTGAAGTTATGTCATATATACACAATATGAATCCTAACATATTAGAGGA
GTGGAATGTTGGTGTTGCACCACCACCTTCAGGAACTTTAGAAGATAGTTATA
GGTATGTACAATCAGAAGCTATTCGCTGTCAGGCTAAGGTAACAACGCCAGAA
AAAAAGGATCCTTATTCAGACTTTTGGTTTTGGGAGGTAAATTTATCTGAAAAG
TTTTCTACTGATTTAGATCAATTTCCTTTAGGTAGAAAGTTTTTACTGCAGGCCG
GGTTGCGTGCAAGGCCTAAACTGTCTGTAGGTAAACGAAAGGCGTCTACAGC
TAAATCTGTTTCTTCAGCTAAACGTAAGAAAACACACAAATAGATGTATGTAGT
AATGTTATGATACATATTTATGTTATTTATTTGTGTACTGTGTTAATAAACTACTTT
TTATATGTTGTGTGTTCTCCATTTTGTTTTTTGTACTCCATTTTGTTTCTAGACCG
ATTTCGGTTGTATCTGGCCTGTTACCAGGTGCATTGGCCATGTTTCCTAACATTT
TGCAAACCTATTCACTTTTTAAATTTATAAATGCAATATGTGCTGCCAACTGTTT
TATGGCACGTATGTTCTGCCAACGTACACTCCCTAATTCCTTTACATAACACAC
ACGCCTTTGCACAGGCATGTGCACAAAGGTTGGCAAAGGTTAGCATATCTCTG
CAGTTACCCATTTCCTTTTTCCTTTTTTTTATGTATGAGTAACTTAATTGTTATAT
GTAATAAAAAAGCTTTTAGGCACATATTTTCAGTGTTGGCATACACATTTACAA
GTTACCTTGGCTTAAACAAGTAAAGTTATTTGTCACTGTTGACACATTACTCAT
ATATATAATTTGTTTTTAACATGCAGGTGGCAACCGAAACCGGTACATAAATCC
TTCTTATTCTTTT-3′ (SEQ ID NO: 10; GENBANK ® Accession No.
A28090.1)

The receptacle with the dry-stored and immobilized nucleic acids can be provided as depicted in item 10 of FIG. 1. In item 20 of FIG. 1, a bodily fluid (e.g., urine or vaginal fluid, either undiluted or diluted with water, for example) is added to the receptacle, causing the first, HRP-labeled complementary nucleic acids to be released from the receptacle wall. In item 30, the released first complementary nucleic acids can bind to nucleic acids from target HPV strains (e.g., high-risk HPV strains) that are present in the sample. The labeled target nucleic acids in the sample then can be captured by the second complementary nucleic acid segments immobilized within the receptacle, as depicted in item 40 of FIG. 1. Specific binding may be enhanced by gently mixing or shaking the vial.

The sample can be washed after the target nucleic acids are captured (Redon et al., DNA Microarrays for Biomedical Research: Methods and Protocols 267-278, 2009), and the fluid can be disposed of (as in item 50 of FIG. 1). A suitable amount of a highly sensitive substrate for HRP, such as TMB, can be added as shown in item 60 of FIG. 1. Oxidation of TMB by HRP present in the receptacle after the wash yields a blue color, indicating the capture of target RNA from an HPV strain that matches the first and second complementary nucleic acids. To interpret the results with the naked eye, a sample (e.g., a drop) of fluid from the receptacle can be transferred onto a support (e.g., a paper strip) integrated with one or more control colors (e.g., a positive control color with or without a negative control color), as depicted in item 70 of FIG. 1. The detection of a test color with an intensity similar to or darker than the positive control can serve as a positive reading, indicating the presence of a target HPV strain.

Any suitable method, including methods known in the art, can be used to reversibly attach the first complementary nucleic acid (e.g., a first sequence from a high risk HPV strain or from another target) to an interior surface of the receptacle. In some embodiments, for example, HRP-modified first nucleic acid sequences that are complementary to sequences from one or more high-risk HPV strains can be vacuum dry-stored (Ramachandran et al.; supra) in sugar alcohol matrices (e.g., sucrose, trehalose, or polyvinyl alcohol (PVA) matrices) on the vial wall to preserve their stability (see, e.g., Stevens et al., supra; and Ivanova and Kuzmina; supra).

Other methods can be used to immobilize the second complementary nucleic acid (e.g., a second sequence from the high risk HPV strain or from another target) within the receptacle. For example, in some embodiments the container bottom can be coated with an agarose film that is activated by NaIO₄ (Afanassiev et al., supra), with the agarose film serving to immobilize second nucleic acid segments that are amino-modified and are complementary to second nucleic acid sequences from the one or more high-risk HPV strains, such that the first and second nucleic acid sequences are complementary to different sequences from the one or more high-risk HPV strains. Activation of the agarose film can lead to formation of aldehyde groups in the agarose, allowing for covalent immobilization of amino groups on the second complementary nucleic acid segments. In some embodiments, a coating of powdered sodium dodecyl sulfate (SDS) on the agarose layer can facilitate lysis of cells in the biological fluid sample.

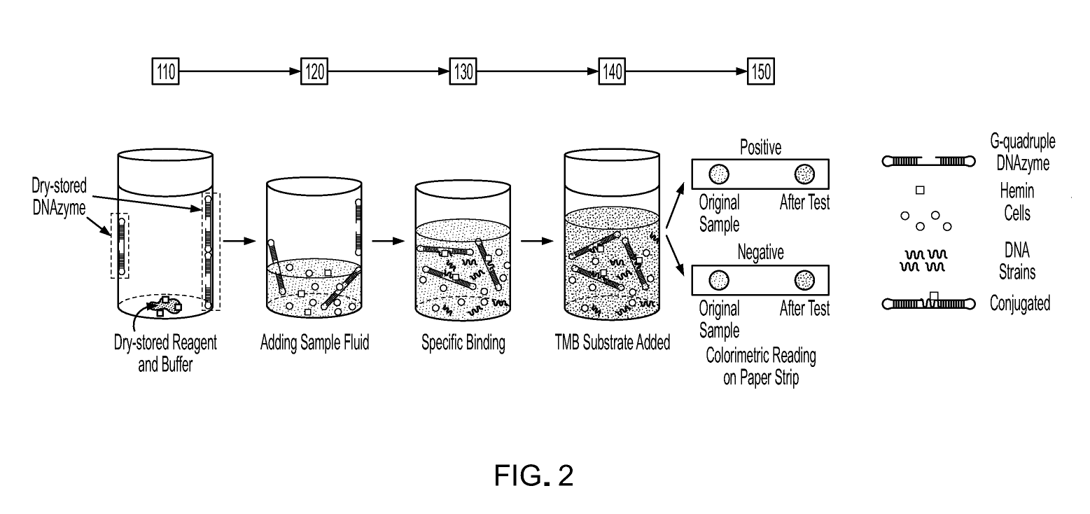

Other mechanisms also can be used to capture target markers in a test receptacle. For example, in some embodiments, a vial-based test can combine in-vial dry reagent storage and the use of DNAzyme (an artificial catalytic DNA; Silverman, Chem Commun 3467-3485, 2008) capable of detecting various targets such as nucleic acids and molecules secreted by cells (see, e.g., Zhou et al., Biosensors Bioelectronics 55:220-224, 2014; Wang et al., J Am Chem Soc 134:5504-5507, 2012; and Ali et al., Angewandte Chemie Int Ed 50:3751-3754, 2011). This approach typically is label-free, which can significantly reduce necessary user steps. DNAzyme and reagents required for the assays, such as a lysis buffer containing, for example, one or more enzymes (e.g., lysozyme, mutanolysin, and/or lysostaphin); one or more alkaline components (e.g., sodium hydroxide); and one or more surfactants (e.g., sodium dodecyl (lauryl) sulfate (SDS); TWEEN® (a polysorbate-type nonionic surfactant formed by ethoxylation of sorbitan before the addition of lauric acid; PLURONIC® (nonionic triblock copolymers composed of a central hydrophobic chain of polyoxypropylene (poly(propylene oxide)) flanked by two hydrophilic chains of polyoxyethylene (poly(ethylene oxide)); TRITON™ X-100 (a nonionic surfactant having the formula C₁₄H₂₂O(C₂H₄O)ₙ (n=9 or 10), with a hydrophilic polyethylene oxide chain and an aromatic hydrocarbon (4-phenyl) lipophilic or hydrophobic group; or any other reagent that can enable the release of genomic materials from the cells), can be concentrated and dry-stored in a sugar matrix (Ramachandran et al., Analyst 139:1456-1462, 2014) in a glass vial, as depicted in step 110 of FIG. 2. Upon rehydration with a bodily fluid, the DNAzyme and reagents can be released, such that cells in the fluid are lysed to release their internal molecules (FIG. 2, step 120). The released DNAzyme can bind to target molecules, whereupon a reagent (e.g., hemin6) released from the vial into the solution can specifically attach to the DNAzyme-target conjugates as depicted in step 130 of FIG. 2. A highly sensitive substrate, such as TMB, can then be added, and the DNAzyme conjugates can cause a catalytic reaction (step 140) that leads to a colorimetric shift toward blue, which can be discernable by the naked eye (step 150).

DNAzyme constructs can be prepared as described elsewhere (see, e.g., Kang et al., Nature Commun 5:5427, DOI: 10.1038/ncomms6427, 2014). In some embodiments, a DNAzyme construct can include a fluorogenic substrate (e.g., 5′-ACTCTTCCTAGCF-rA-QGGTTCGATCAAGA-3′; SEQ ID NO:11, where (“F” indicates fluorescein-dT, “rA” indicates riboadenosine, and “Q” indicates dabcyl-dT), and a catalytic sequence (e.g., 5′-CACGGATCCTGACAAGGATGTGTGCGTTGTCGAGACCTGCGACCGGAACACT ACACTGTGTGGGATGGATTTCTTTACAGTTGTGTGCAGCTCCGTCCG-3′; SEQ ID NO:12). In some embodiments, where a DNAzyme is generated for an HPV 16 target, the fluorogenic substrate and the catalytic sequence can be covalently joined (e.g., through template-mediated enzymatic ligation) using a template marker sequence such as 5′-GCACAGGGACATAATAATGGCATTTGTTGGGGTAACCAACTATTTGTTACTG TTGTTGATACTACACGCAGTACAAATATGTCATTATGTGCTGCCATATCTACTTC AGAAACTACATATAAAAATACTAACTTTAAGGAGTACCTACGACATGGGGAGG AATATGATTTACAGTTTATTTTTCAACTGTGCAAAATAACCTTAACTGCAGACG TTATGACATACATACATTCTATGAATTCCACTATTTTGGAGGACTGGAATTTTGG TCTACAACCTCCCCCAGGAGGCACACTAGAAGATACTTATAGGTTTGTAACCC AGGCAATTGCTTGTCAAAAACATACACCTCCAGCACCTAAAGAAGATGATCCC CTTAAAAAATACACTTTTTGGGAAGTAAATTTAAAGGAAAAGTTTTCTGCAGA CCTAGATCAGTTTCCTTTTGGACG-3′ (SEQ ID NO:13), where the template marker sequence of SEQ ID NO:13 is a target-specific sequence for HPV 16. The template marker sequence can be replaced with any other sequence to be specific for a different marker (e.g., a marker for a different HPV type, another virus, a bacterium, or a mammalian cell).

This concept can be tailored to cater to different clinical needs. For instance, when identifying low-abundance targets from complex clinical samples, a DNAzyme and its relevant chemistry can be designed to generate a fluorescent signal (Ali et al., supra; and Kang et al., Nature Commun 5:5427, 2014) that offers a higher resolution. In some embodiments, the fluorescent signal can be detected by a smart phone with a particular lens and filter attached to its camera (Zhu et al., Analyst 137:2541-2544, 2012). With an image analysis smart phone app, a user can obtain results with just a few taps on the screen.

The receptacles used in the products and methods described herein can be of any suitable size (e.g., 0.5 to 5 mL, 1 to 10 mL, 5 to 20 mL, 20 to 50 mL, 1 mL, 5 mL, 10 mL, 20 mL, 25 mL, or 50 mL), and can be made of a material to which nucleic acids and support polymers (e.g., agarose) can be reversibly or permanently attached. In general, a receptacle can be large enough to contain a typical sample of body fluid obtained from a subject, such as a 0.5 to 10 mL (e.g., 0.5 to 1 mL, 1 to 3 mL, 3 to 5 mL, 5 to 10 mL, 0.5 mL, 1 mL, 2 mL, 2.5 mL, 3 mL, 5 mL, 7.5 mL, or 10 mL) sample of urine or vaginal fluid, for example, and then to contain a suitable amount of TMB or other detectable substrate (e.g., 0.5 to 10 mL, 1 to 5 mL, 5 to 10 mL, 0.5 mL, 1 mL, 5 mL, or 10 mL of substrate). In some embodiments, the test kits provided herein can utilize commercially available glass vials (e.g., 5 to 10 mL, 10 to 20 mL, or 20 to 50 mL glass vials), which can largely reduce the need for microfabrication as compared with other point-of-care diagnostic devices.

The platform described herein can be used for detection of a wide variety of viral, bacterial, and cellular markers, and can be used with any bodily fluid sample (e.g., urine, sputum, blood, plasma, serum, cerebrospinal fluid, lymph fluid, or synovial fluid) that may contain free-floating virus particles, bacteria, or other cells of interest, for example. These materials and methods can have a high clinical value, in that they can be used to detect acute agents (e.g., acute viral agents) in vulnerable subjects that would benefit from real-time testing and immediate administration of targeted treatment.

In some embodiments, the tests can be adapted to detect various viral genetic signatures, including genes that provide resistance to antiviral treatments. The materials and methods also can be used to monitor the status of infected patients. In some cases, for example, the materials and methods provided herein can be used to detect viral agents linked to respiratory infections. Real-time, rapid detection of viral agents that cause respiratory infection can be useful to determine whether immediate treatment should be pursued, particularly for vulnerable patients such as infants, the elderly, or those who are immunocompromised. Typical tests for influenza and respiratory syncytial virus (RSV) can require 24 hours to retrieve a result. While this time frame may be acceptable for healthy adults, members of more vulnerable populations may require hospitalization in case life-threatening complications develop during the time period before results are obtained. The real-time test provided herein can allow the decision whether to hospitalize to be made immediately, and targeted treatment can be administered at the same clinical visit. Moreover, the assayed markers also can include one or more genes that confer resistance to known antiviral treatments, saving time in attempting a treatment stream that will fail.

In some cases, a test for detecting influenza or RSV can, in some embodiments, be similar to that for detecting HPV, except that instead of urine or cervical fluid, sputum can be used as the biological fluid sample. The sputum can be directly expelled by the subject into a vial containing an immobilized, amino-modified nucleic acid (e.g., HRP-modified RNA) complementary to one or more RSV markers. TMB subsequently added to the vial will turn blue if the marker(s) are present.

Other viral agents can be screened for in blood samples using the methods and materials provided herein. These include, without limitation, hepatitis A, B, and C (e.g., to determine treatment for acute hepatitis), herpes simplex virus (e.g., to determine treatment of aseptic meningitis), cytomegalovirus (e.g., to determine treatment for infectious mononucleosis), human immunodeficiency virus (HIV) (e.g., for post-exposure prophylactic monitoring), rabies (e.g., to determine treatment for encephalitis), and varicella-zoster virus (e.g., to determine treatment for chickenpox).

In addition, the tests provided herein can be used to assay for the presence of bacterial agents in a biological fluid sample. For example, the disclosed materials and methods can be used to detect bacterial endotoxins and/or exotoxins, including those produced by members of the Escherichia, Salmonella, Shigella, Pseudomonas, Borderella, and Vibrio genera, which often are responsible for acute illness. In such cases, a lipopolysaccharide (LPS) probe for the LPS signature of a bacterial agent, rather than a nucleic acid probe for the genetic signature of the agent, can be linked to HRP. As for methods that involve detecting a genetic signature, an added substrate (e.g., TMB) can generate a visible signal if the matching LPS signature is present in the test sample. It is noted that methods utilizing LPS probes would likely not include using SDS to facilitate cell lysis.

Further, it is noted that the platform provided herein may be useful for detecting bacteria or other cells (e.g., eukaryotic cells) directly. Such assays can utilize a sealed vial that can be mixed (e.g., vortexed) without disrupting conjugates between the marker to be detected and the probe(s) within the vial. For assays designed to detect bacteria, SDS can be used to lyse at least some of the bacterial cells, and the lysate can be added to a vial in which nucleic acids with sequences complementary to, for example, the 16S RNA of the target bacteria have been dry-stored and immobilized. Bacterial detection using DNAzyme-based methods may not require dry-storage of anything other than the DNAzyme, since bacterial lysates can specifically react with an immobilized DNAzyme designed for the target bacteria. Methods of detecting eukaryotic (e.g., endothelial) cells can utilize a vial in which DNA complementary to a sequence from the target cells has been immobilized. The rest of the procedure and detection mechanism can be carried out as described herein for bacteria or virus particles.

The methods and materials described herein also can utilize immunoglobulins of various types (e.g., IgA, IgD, IgE, IgG, and IgM) for real-time testing of exposure to pathogens, immunization status, and allergens. For such applications, the SDS used for cell lysis may be replaced with a detergent such as TRITON® X-100 or TWEEN®, for example. Antibodies (e.g., anti-IgA, anti-IgD, anti-IgE, anti-IgG, or anti-IgM antibodies), can be immobilized within a glass vial using, for example, a method that relies on a condensation reaction between an aldehyde group on the antibodies and the hydrazide group on the modified glass surface, as described elsewhere (Gering et al., J Colloid Interface Sci 252(1):50-55, 2002). HRP-functionalized secondary antibodies can be dry-stored in a sugar matrix on another interior surface of the vial. A fluid sample containing target molecules can be added to the vial, where the target molecules can be captured by the pre-immobilized antibodies, and the dry-stored, HRP-functionalized antibodies can be rehydrated by the fluid and released from the sugar matrix. The target molecules then can become sandwiched between the pre-immobilized antibodies and the HRP-functionalized antibodies. At this point, the vial can be washed (e.g., three times), and the TMB substrate can be added.

In addition, the materials and methods provided herein can be used to test for free-floating genetic markers in the blood or another bodily fluid, expanding blood liquid biopsies to a real-time application. For such methods, first and second nucleic acid probes, or a nucleic acid probe such as a DNAzyme and a reagent that can specifically bind to the DNAzyme when it is recognized by the genetic material of interest, can be positioned within a receptacle (e.g., where a first, HRP labeled nucleic acid is reversibly attached to an inner surface of the receptacle, and a second nucleic acid is immobilized on a second inner surface of the receptacle), without a reagent for lysing cells. If the genetic marker of interest is present within a sample added to the receptacle, it can bind to the first and second nucleic acid probes, resulting in generation of a signal that is retained within the receptacle after washing, thus indicating a positive result.

As noted herein, the materials described can be included in kits for detecting selected markers of, for example, particular viruses or bacteria. A kit can include a receptacle having one or more nucleic acid, polypeptide, or LPS probes reversibly and/or permanently immobilized on one or more interior surfaces. In some cases, a kit can include a receptacle having a nucleic acid probe and a reagent reversibly immobilized on one or more interior surfaces. In some embodiments, a kit also can include a control component showing a positive control, a negative control, or both. The control component also can include a portion for receiving a test sample, which may facilitate comparison to the positive and/or negative controls. The support can be, for example, a test paper strip. In some cases, the support (e.g., the test paper strip) included with the kits provided herein and integrated with the control(s) can be calibrated to compensate for background noise, facilitating interpretation of the result.

TABLE 1 provides the estimated cost for one embodiment of a test as provided herein (based on Sigma Aldrich products unless otherwise stated).

TABLE 1
Cost per test

| | |
|---|---|
| Components/test | Cost/test |

Test vial preparation

| | |
|---|---|
| Vial (10 mL) | $0.28 (Wholesale-vials) |
| Agarose (3 mg) | $0.01 |
| NaIO₄ (20 mg) | $0.04 |
| Amino-modified RNA | $0.075 (Integrated DNA |
| (1 nmole total) | technologies) |
| HRP-modified RNA | $0.65 (Integrated DNA |
| (0.5 nmole total) | Technologies) |
| SDS (10 mg) | $0.01 |

Dry reagent storage

| | |
|---|---|
| Sucrose (30 mg) | $0.01 |
| Trehalose (30 mg) | $0.13 |
| PVA (100 mg) | $0.02 |
| Desiccator | $0.01 (Science Lab Supplies) |

Colorimetric quantification

| | |
|---|---|
| TMB substrate (1 mL) | $0.36 (Thermo Fisher) |
| Liquid dropper | $0.01 |
| Paper strip (1″ × 3″) | $0.01 (VWR) |
| Estimated total | $1.615 |

The invention will be further described in the following example, which does not limit the scope of the invention described in the claims.

### EXAMPLE

A test platform is developed using high-risk HPV strains (HPV types 16, 18, 31, 33, 35, 39, 45, 51, 52, 56, 58, 59, and/or 68) and low risk HPV strains (HPV types 6, 11, 40, 42, 43, 44, 53, 54, 61, 72, 73, and/or 81; de Sanjose et al., The Lancet Oncol 11(11):1048-1056, 2010) added to sterile saline as substrates at variable concentrations consistent with what would be expected in a biological sample (<1 femtomole, 600,000,000 copies (Denny et al., JAMA 294(17):2173-2181, 2005)). The sensitivity and specificity of the test are calculated and adjusted until the levels displayed by the clinical standard test are reached. This demonstrates efficacy and performance of the low-cost platform as compared to standard of care tests.

In particular, silanized clear glass vials (20 mL, Thermo Fisher Scientific) are obtained. One percent (1%) agarose in purified water is poured into the vial at 70° C., such that the bottom interior surface of the vial is covered. After agarose gelling occurs, the vial is dried in air. 20 mM NaIO₄ is prepared under suitable conditions (e.g., in a chemical hood that can vent hazardous gases). The NaIO₄ solution is added into the agarose-coated vials at room temperature for 30 minutes for agarose activation. The activation leads to the formation of aldehyde groups in the agarose, enabling the covalent binding of amino groups. DNA is suspended in spotting buffer (0.15 M NaCl, 0.1 M NaHCO₃, pH8.5), and pipetted onto the agarose film in the vial. The vial is incubated in a humid incubator overnight, and dried at room temperature. Drops of sodium borohydride solution (50 mg NaBH₄ in 30 mL Phosphate Buffer Saline (PBS) with 10 mL ethanol) is pipetted into the vial under suitable conditions (e.g., in a chemical hood). After five minutes, the vial is washed three times with 0.2% sodium dodecyl sulfate (SDS) for 2 minutes, then washed twice in distilled water for 1 minute, and dried at room temperature.

Amino-modified complementary DNA segments are immobilized on the activated agarose film via NH₂ groups, and the vial is dried at room temperature. Horseradish peroxidase (HRP)-modified DNA segments are added to sugar alcohol matrices (sucrose, trehalose, polyvinyl alcohol (PVA)) to preserve their stability, and then added into the vial, which is then vacuum-dried at 30° C. for 2 hours. The product is stored in a dry place at room temperature.

Patients undergoing HPV screening as clinically indicated (based on reflex HPV test if atypical cells of unknown significance (ASCUS) are found by Pap Smear) are enrolled in the test. The same number of age-matched patients (control cohort) with low risk for cervical cancer and undergoing routine Pap Smear testing also are enrolled. This tests the detection kit with real samples and real patients, to validate performance in the field and ease of interpretability by users. An additional urine sample from test subjects is obtained for laboratory testing and optimization if such becomes necessary.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A kit comprising:
(a) a receptacle for receiving a biological fluid sample, the receptacle having a first nucleic acid reversibly attached to a first interior surface and a second nucleic acid immobilized on a second interior surface, wherein the first nucleic acid is complementary to a first nucleic acid sequence of a selected marker, and wherein the second nucleic acid is complementary to a second nucleic acid sequence of the marker; and
(b) a substrate comprising a first portion with an integrated positive control result and a second portion for receiving a test fluid.

2. The kit of claim 1, wherein the receptacle is a glass vial.

3. The kit of claim 2, wherein the glass vial has a volume of 10 to 20 mL.

4. The kit of claim 2, wherein the first nucleic acid is dry-stored on the first interior surface.

5. The kit of claim 2, wherein the second nucleic acid is coupled to an agarose film on the second interior surface.

6. The kit of any one of claims 1 to 5 claim 1, wherein the first nucleic acid is coupled to horseradish peroxidase (HRP).

7. The kit of claim 1, wherein the marker is from one or more high-risk human papillomavirus (HPV) strains.

8. The kit of claim 7, wherein the one or more high-risk HPV strains comprise one or more of HPV 16, HPV 18, HPV 31, HPV 33, HPV 35, HPV 39, HPV 45, HPV 51, HPV 52, HPV 56, HPV 58, HPV 59, and HPV 68.

9. The kit of claim 1, wherein the substrate comprises a paper strip.

10-18. (canceled)

19. A method for determining that a biological fluid contains a selected marker, comprising:
(a) providing a receptacle for receiving the biological fluid, the receptacle having a first nucleic acid reversibly attached to a first interior surface and a second nucleic acid immobilized on a second interior surface, wherein the first nucleic acid is complementary to a nucleic acid sequence of the marker and is labeled with a means for visual detection, and wherein the second nucleic acid is complementary to a second nucleic acid sequence of the marker;
(b) placing a sample of the biological fluid into the receptacle, such that the first nucleic acid is released from the first interior surface, and the first and second nucleic acids hybridize to the first and second complementary nucleic acid sequences of the marker, such that the first nucleic acid becomes attached to the second interior surface via the marker and the second nucleic acid;
(c) removing the biological fluid from the receptacle;
(d) washing the interior of the receptacle to remove residual biological fluid;
(e) placing a substrate that interacts with the means for visual detection into the receptacle, such that the means for visual detection can act on the substrate to generate a signal; and
(f) visually inspecting the vial containing the substrate, or a sample of the substrate, to determine that the signal is present, thus indicating the presence of the high-risk HPV strain in the biological fluid.

20. The method of claim 19, wherein the receptacle is a glass vial.

21. The method of claim 20, wherein the glass vial has a volume of 10 or 20 mL.

22. The method of claim 20, wherein the first nucleic acid was dry-stored on the first interior surface prior to providing the receptacle.

23. The method of claim 20, wherein the second nucleic acid was coupled to an agarose film on the second interior surface prior to providing the receptacle.

24. The method of claim 19, wherein the means for visual detection is HRP.

25. The method of claim 24, wherein the substrate is tetramethylbenzidine (TMB).

26. The method of claim 19, wherein the marker is from one or more high-risk human HPV strains.

27. The method of claim 26, wherein the one or more high-risk HPV strains comprise one or more of HPV 16, HPV 18, HPV 31, HPV 33, HPV 35, HPV 39, HPV 45, HPV 51, HPV 52, HPV 56, HPV 58, HPV 59, and HPV 68.

28. The method of claim 19, wherein the biological fluid comprises urine or vaginal fluid.

29-38. (canceled)


SEQUENCE LISTING (13 sequences)

SEQ ID NO: 1 - DNA, 452 bp, Human papillomavirus type 16
gcacagggacataataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacccaggcaattgcttgtcaaaaacatacacctccagcacctaaagaagatgatccccttaaaaaatacactttttgggaagtaaatttaaaggaaaagttttctgcagacctagatcagtttccttttggacg

SEQ ID NO: 2 - DNA, 1386 bp, Human papillomavirus type 18
atggtatcccaccgtgccgcacgacgcaaacgggcttcggtaactgacttatataaaacatgtaaacaatctggtacatgtccacctgatgttgttcctaaggtggagggcaccacgttagcagataaaatattgcaatggtcaagccttggtatatttttgggtggacttggcataggtactggcagtggtacagggggtcgtacagggtacattccattgggtgggcgttccaatacagtggtggatgttggtcctacacgtcccccagtggttattgaacctgtgggccccacagacccatctattgttacattaatagaggactccagtgtggttacatcaggtgcacctaggcctacgtttactggcacgtctgggtttgatataacatctgcgggtacaactacacctgcggttttggatcccacaccttcgtctacctctgtgtctatttccacaaccacctttaccaatcctgcattttctgatccgtccattattgaagttccacaaactggggaggtggcaggtcctgtatttgttggtacccctacatctggaacacatgggtatgaggaaatacccctacaaacatccgcttcttctggtacgggggaggaacccattagtagtaccccattgcctactgtgcggcgtgtagcaggtccccgcctttacagtagggcctaccaacaagtgtcagtggctaaccctgagtttctcccacctccatcctcccccattacatatgacaacccggcctttgagcctgtggacactacattaacatttgatcctcgtagtgatgttcctgattcagattttatggatattatccgtctacataggcctgctccaacatccacccgtgggactgttcgctttagtagattaggtcaacgggcaactatgtttacccgcagcggtacacaccccggtgctagggttcacttttatcatgatataagtcctattgcaccttccccagaatatcctgaactgcagcctttagtatctgccacggaggacaatgacttgtttgatatatatgcagatgacatggaccctgcagtgcctgtaccatcgcgttctactacctcctttgcatcctttaaatattcgcccactatatcttctgcctcttcctatagtaatgtaacggtccctttaacctcctcttgggatgtgcctgtatacacgggtcctgatattacattaccatctactacctctgtatggcccattgtatcacccacggcccctgcctctacacagtatattggtatacatggtacacattattatttgtggccattaccttattttattcctaagaaacgtaaacgtgttccctatttttttgcagatggctttgtggcggcc

SEQ ID NO: 3 - DNA, 852 bp, Human papillomavirus type 31
ttatgtatgcgtgtgtacttgtatatatgtatagtatgttatgtgtgtatgtatgctatgtatgttaataaatatgtgtatacctgtgtgtgttgtgtatgttgtccttatatacacccttactattactattttataaactattgttcctacttgttcctgctcctcccaatagtcatgtacttatttctgcctataatttaggtgtcacgccatagtaaaagttgtacacccggtccgttttttgcaactaaagctactccattttgattttatgcagccattttaaatccctaaccgttttcggttgcattgtttaaacatgctagtacaactatgctgatgcagtagttctgcggtttttggtttcctgaatactagtttttgccaacattctggcttgtagtttcctgcctaacacaccttgccaacatataatccagtccaactttgcaattatactatgaatcatgtttgtttaaatacaactgtagttcaactatgtgtcatgcacatatattatattatcctacacaccttaaactgcttttaggcacatattttgtagattatctatatccttgattgcagtgctggcttttgcacatgtttaaactgccaaggttgtgtcatgcattataaataagttgtatgttactcatataattaattgcatataggtattacaccgttttcggttacagttttacaagcaattgttctttttatacttaataataataatcttagtataaaaaagtagggagtgaccgaaagtggtgaaccgaaaacggttggtatataaagcacatagtattttgtgcaaacctacagacgccatgttcaaaaatc

SEQ ID NO: 4 - DNA, 758 bp, Human papillomavirus type 33
atgtttcaggacactgaggaaaaaccacgaacattgcatgatttgtgccaagcattggagacaactatacacaacattgaactacagtgcgtggaatgcaaaaaccctttgcaacgatctgaggtatatgattttgcatttgcagatttaacagttgtatatagagagggaaatccatttggaatatgtaaactgtgtttgcggttcttatctaaaattagtgaatatagacattataattattctgtatatggacatacattagaacaaacagttaaaaaacctttaaatgaaatattaattaggtgtattatatgtcaaagacctttgtgtcctcaagaaaaaaaacgacatgtggatttaaacaaacgttttcataatatttcgggtcgttgggcagggcgctgtgcggcgtgttggaggtcccgacgtagagaaactgcactgtgacgtgtaaaaacgccatgagaggacacaagccaacgttaaaggaatatgttttagatttatatcctgaaccaactgacctatactgctatgagcaattaagtgacagctcagatgaggatgaaggcttggaccggccagatggacaagcacaaccagccacagctgattactacattgtgacctgttgtcacacttgtaataccacagttcgtttatgtgtcaacagtacagcaagtgacctacgaaccatacagcaactacttatgggcacagtgaatattgtgtgccctacctgtgcacaactataa

SEQ ID NO: 5 - DNA, 15 bp, Human papillomavirus type 39
  misc_feature (7)..(9): A, C, T, or G
ggtacannntgttct

SEQ ID NO: 6 - DNA, 8039 bp, Human papillomavirus type 45
gaattccaggcctaatttgagatgtgagttgtatctgtaacccagtgcccttgaaggtgagggcaggcactcagcagcctctccaggaaggctcacatcctgggaggactcactgattagttctattgtgttcatttgtctgtgtcttaagctgaagggaagagttaaaaccaagcctttccctgggggtctggatgaacagaactcaacccaaagagtggcattgccttgtccttggagcagggagctgggaccccccttggactttgaaaaccagtgttttcagaatgcaggtggataacaagcctaaatttacttctgggctgaggagagatctttgaggctcctggaaggaaacttggtgataagcctccagtttgaaacggctctgtccctttaatgtctgtgccttgacagcttttggtgaggaagcacttccttccaacagctgtcttcttggcagaaaaccaaaacattggcttaaagggacccacagactggaacagcctcacatttcggctttagaacaaatcccacaattgttcagctttccggtccccttcagatcaagcagaagatatgttttgattttcatgcttgtattttaaacaataattttctaccccagcgtggtagtcaatgaggagagaggggaagaatgcgcacatgatgctacacgtttctgttgttgctgttattattggtggctttgaggagagctgctcccatttggggtttataccaactgtggattatggctttgtcattaagatttgatctttgttaaatgaaaaactgtttattgtataaaactcaggtttgtggacgaaaagttgttttttttcttcagttaattaaattgttcctcaagtttgtttaaggacttaaaatcaaacacaaccatgtgtaaactgctaaatgaggctcctaaaatgagaggcctcaactctttaagtgtggagctagaaatgtaaataagtccacagggcagactggtgattatgataaaagctaccatttactgagcatctgtctactaggctcagctctatgctaagtctacatgttatctgtcaaagtggtatcatccccatttaatagctgaggaaacagaggcttagaaaggctgggtaacttgaccagggtcatgcaactagtctgcggtggagccaggattctgtctgaccctaaaggccaagttctttatatttatttctaccacctgctaaagtcttgaatggaggctgaaagcacagttggggtatggggaagaaaaatatatatacatacatatatgtatatgtatgtatgtatgtatggggggttgttttgtttttgtttttgataaggagttttgctcttgttgcccaggctggagtgcagtggtatgatctgggctcactgcaacctccgcctcccgggttcaagtcattctcctgcctcagcctcccgagtagctgggattaccggagcatgccaccacacccagcaaagttttgtatttttagtagagacagggtttcaccatgttggccaggctgatcttgaactcctcatctcaggtgatctgcccgcctccgcttcccaaagtgctgggattacaggtgtgagtcaccgcgtccggcctacagatatatttaatttaaagagatctaaaacaaatacaaaactgtccacatctatgttgatggacccataaaaatagcagtctgccagggtctgccggaagagacagataagcatacatattaacatggatatatatgtgaatttcattcaaatggttctcacatgagagtaactagcatctttctctcagatgatgaagatgatgaagaggaagatgaagaggaagaaatcgacgtggtcactgtggagaagcggcgttcctcctccaacaccaaggctgtcaccacattcaccatcactgtgcgtcccaagaacgcagccctgggtcccgggagggctcagtccagcgagctgatcctcaaacgatgccttcccatccaccagcagcacaactatgccgccccctctccctacgtggagagtgaggatgcacccccacagaagaagataaagagcgaggcgtccccacgtccgctcaagagtgtcatccccccaaaggctaagagcttgagcccccgaaactctgactcggaggacagtgagcgtcgcagaaaccacaacatcctggagcgccagcgccgcaacgaccttcggtccagctttctcacgctcagggaccacgtgccggagttggtaaagaatgagaaggccgccaaggtggtcattttgaaaaaggccactgagtatgtccactccctccaggccgaggagcaccagcttttgctggaaaaggaaaaattgcaggcaagacagcagcagttgctaaagaaaattgaacacgctcggacttgctagacgcttctcaaaactggacagtcactgccactttgcacattttgattttttttttaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttctttttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggaagttcatacctaagtactgtaataatacctcaatgtttgaggagcatgttttgtatacaaatatattgttaatctctgttatgtactgtactaattcttacactgcctgtatactttagtatgacgctgatacataactaaatttgatacttatattttcgtatgaaaatgagttgtgaaagttttgagtagatattactttatcactttttgaactaagaaacttttgtaaagaaatttactatatatatatgcctttttcctagcctgtttcttcctgttcatgtatttgttcatgtttggtgcatagaactgggtaaatgcaaagttctgtgtttaatttcttcaaaatgtatatatttagtgctgcatcttatagcactttgaaatagatttagtagattgggtcaaagggcaaccatgtttacacgtagtggtaaacaaatagggggtagggtacatttttaccatgatataagccccattgctgctacagaggaaattgaattgcagcctttacttagtgctacagatgatagtgacctgtttgatgtatatgcagactttccacctcctgcgtccactacacctagcactataaacaaatcatttacatatccaaagtattcattgaccatgccttccactgctgcatcctcttacagtaatgttacagtaccattaacatctgcatgggatgtacctatatatactggcccggacattatattgccatcccatactcctatgtggcctagtacatctcctaccaatgctgccacctccacctatataggtattcatggcacacaatattatttatggccatggtattattattttcctaaaaaacgtaaacgtattccctatttttttgcagatggctttgtggcggcctagtgacagtacggtatatcttccaccaccttctgtggccagagttgtcaacactgatgattatgtgtctcgcacaagcatattttaccatgcaggcagttcccgattattaactgtaggcaatccatattttagggttgtacctagtggtgcaggtaataaacaggctgttcctaaggtatccgcatatcagtatagggtgtttagagtagctttgcccgatcctaataaatttggattacctgattctactatatataatcctgaaacacaacgtttggtttgggcatgtgtaggtatggaaattggtcgtgggcagcctttaggtattggcctaagtggccatccattttataataaattggatgatacagaaagtgctcatgcagctacagctgttattacgcaggatgttagggataatgtgtcagttgattataagcaaacacagctgtgtattttaggttgtgtacctgctattggtgagcactgggccaagggcacactttgtaaacctgcacaattgcaacctggtgactgtcctcctttggaacttaaaaacaccattattgaggatggtgatatggtggatacaggttatggggcaatggattttagtacattgcaggatacaaagtgcgaggttccattagacatttgtcaatccatctgtaaatatccagattatttgcaaatgtctgctgatccctatggggattctatgtttttttgcctacgccgtgaacaactgtttgcaagacatttttggaatagggcaggtgttatgggtgacacagtacctacagacctatatattaaaggcactagcgctaatatgcgtgaaacccctggcagttgtgtgtattccccttctcccagtggctctattactacttctgattctcaattatttaataagccatattggttacataaggcccagggccataacaatggtatttgttggcataatcagttgtttgttactgtagtggacactacccgcagtactaatttaacattatgtgcctctacacaaaatcctgtgccaaatacatatgatcctactaagtttaagcactatagtagacatgtggaggaatatgatttacagtttatttttcagttgtgcactattactttaactgcagaggttatgtcatatatccatagtatgaatagtagtatattggaaaattggaattttggtgtacctccaccacctactacaagtttagtggatacatatcgttttgtgcaatcagttgctgttacctgtcaaaaggatactacacctccagaaaagcaggatccatatgataaattaaagttttggactgttgacctaaaggaaaaattttcctccgatttggatcaatatccccttggtcgaaagtttttagttcaggctgggttacgtcgtaggcctaccataggacctcgtaagcgtcctgctgcttccacgtctactgcatctaggcctgccaaacgtgtacgtatacgtagtaaaaaataatatgttagcacatatatgtatgtttgtatgtatggttttgtatgttgtatgtatgtatgtatttgtgtgatatattactgtattttgtttgtttgcgtgcgtgtatgtatgaatgtgccttgtggcatgtatggtgttactgtacataattgtggtattaaataaagtatgatactagtgttgtgtagggttgcacccttgtgagtaacactactatttgtgtgtatgtgtattgctttataccctatattccttcctgtatttcaagttataaacttgcatactacacagcatccattttacttataatcctccattttgctgtgcaaccgatttcggttgcctgtggcttatgtttgaccttttaaacataatacttaaactggcacatttacaacccctacatagtttaaactactggcgcgccttcttggcgtacatgtggcacacctggtattagtcattttcctgtccaggtgcactaaaacaatggcttgcacaactgtatccacaccctatgtaataaaactgcttttaggcacatattttagtctgtttttacctgtgctaattgtataattggcatgtagaaccactttcttatccaacaatctgtctacttgttacatgaactataaactgactcacttatacatacatagtttatgcaaccgaaaaaggttgggccctataacacataccttttcttaatacttttaacaatgatactacataaaaaagggtgtaaccgaaaacggttgcaaccaaaaacggtgcatataaaaactttgtggaaaagtgcattacaggatggcgcgctttgacgatccaacgcaacgaccctacaagctaccagatctgtgcacagaattgaatacatcactacaagacgtatctattgcctgtgtatattgcaaagcaacattggaacgcacagaggtatatcaatttgcttttaaagatttatttatagtgtatagagactgtatagcatatgctgcatgccataaatgtatagacttttattccagaattagagaattaagatattattcaaactctgtatatggagagacactggaaaaaataactaatacagagttgtataatttgttaataaggtgcctgcggtgccagaaaccattgaacccagcagaaaaacgtagacaccttaaggacaaacgaagattccacagcatagctggacagtaccgagggcagtgtaatacatgttgtgaccaggcacggcaagaaagacttcgcagacgtagggaaacacaagtatagcaataagtatgcatggaccccgagcaacactgcaagaaattgtattgcatttggaacctcagaatgaattagatcctgttgacctgttgtgttacgagcaattaagcgagtcagaggaggaaaacgatgaagcagatggcgttagtcatgcacaactaccagcccgacgagccgaaccacagcgtcacaaaattttgtgtgtatgttgtaagtgtgacggcagaattgagcttacagtagagagctcggcagatgaccttagaacactacagcagctgtttttgagcaccttgtcctttgtgtgtccgtggtgtgcaactaaccaataatctacaatggcggatccagaaggtaccgacggggagggaacggggtgtaatggctggttttttgtagaaacaattgtagagaaaaaaacaggggatgtaatatcagatgatgaggatgaaaccgcaacagatacagggtcggatatggtagattttattgacacacaattatccatttgtgaacaggcagagcaagagacagcacaggcattgttcgatgcgcaggaagttcagaatgatgcacaggtgttggatcttttaaaacgaaactttgcaggaggcagcaaggaaaacagtccattaggggagctactctggaggctgaggcatgcgaatctcttgaacctgggaggcggaggttgcagtgagccagtatggtgccactgcactccagcctgggcgacagagtgactctgtctcaaaaaaaagaaaaagaaaagaaatcagccaggcatagtcccagctaccagggaggttgaggcaagagaattgcttgaacccaggaggcggaggttgcggtgagctgagactctgtctcaaaacacacacatacacacatacatacacacacacacacacacacacacacacaccctgaatgggtgggtttaaatgaagattaaatccaagagggaaaataggtaaattggaatatcaagaaatgatctagagtgcagcacagaaacaaagaaagaaaggctaaaagatgtgaaaaagtgggtgagaaggactaaaacatgctcagactggagttccgttaggacaggagtgagaaaatgccctaactgacgccacaggagcagcagagacgcatcctcactggacctcacccaaatcgcaaattcatgagcaaaacaaatcattgttgttttaggccactaagtttaggtagttttttatgaagcagtagataactggaataagttgtgataccaggagtgtgatgtcgccatattaaaacacacacacacacacactcacacacatacaacctaaaaatgtaacattggctttgggaccagatggtgagtggaagccaaaagggcttgcagaggctgctggtgaggacttaaaagaaatgcaagagaggcttattggaatctaga

SEQ ID NO: 7 - DNA, 7812 bp, Human papillomavirus type 51
aacaattatcttgtaaaaactagggtgtaaccgaaaagggttatgaccgaaaacggtgcatataaaagtgaagaggtaaaagtatagaagaacaccatgttcgaagacaagagggaaagaccacgaacgctgcatgaattatgtgaagctttgaacgtttctatgcacaatatacaggtagtgtgtgtgtattgtaaaaaggaattatgtagagcagatgtatataatgtagcatttactgaaattaagattgtatatagggataataatccatatgcagtatgcaaacaatgtttactgttttattcaaaaattagagagtatagacgttatagcaggtctgtgtatggtactacattagaggcaattactaaaaaaagcttatatgatttatcgataaggtgtcatagatgtcaaagaccacttgggcctgaagaaaagcaaaaattggtggacgaaaaaaaaaggttccatgaaatagcgggacgttggacggggcaatgcgctaattgctggcaacgtacacgacaacgtaacgaaacccaagtgtaataaagccatgcgtggtaatgtaccacaattaaaagatgtagtattgcatttaacaccacagactgaaattgacttgcaatgctacgagcaatttgacagctcagaggaggaggatgaagtagataatatgcgtgaccagctaccagaaagacgggctggacaggctacgtgttacagaattgaagctccgtgttgcaggtgttcaagtgtagtacaactggcagtggaaagcagtggagacactcttcgcgttgtacagcagatgttaatgggcgaactgagcctggtttgcccgtgttgtgcgaacaactagcaacggcgatggactgtgaaggtacagaggatgagggggcggggtgtaatgggtggttttttgttgaagcaatagtagaaaaaaaaacaggagatactgtttcggatgatgaggatgaaaatgcagatgatacaggatctgatttaataaactttatagatagtgaaactagtatttgcagtcaggcggaacaggagacagcacgggcgttgtttcaggcccaagaattacaggcaaacaaagaggctgtgcatcagttaaaacgaaagtttctagtcagcccgcgaagcagcccattaggagacattacaaatcaaaacaacacacacagccatagtcaggcaaacgagtcacaagttaaaaggagattactggacagttatccggacagcggatatggcaatacacaagtggaaactgtggaagcaacgttgcaggtagatgggcaacatggcggttcacagaacagtgtgtgtagtagcggggggggcagtgttatggatgtggaaacaacagaaagctgtgaaaatgtagaactaaacagtatatgtgaagtattaaaaagcagtaatgcaaaagcaacgttaatggcaaaattcaaagagttgtatggtattagttataatgagttggtacgggtgtttaaaagtgataaaacatgttgtatagattgggtttgtgcattgtttggcgtttccccaatggtagcagaaaatttaaaaacactaattaagccattttgcatgtactaccatatacaatgtttatcatgtgattggggcaccattgtattaatgctaattaggttttcatgtgcaaaaaacagaacaacaattgctaagtgtttaagtacattagtaaatatcccacaatcacaaatgtttatagaaccaccaaaattacgtagtacacctgtggcattatatttttatagaacaggcatatcaaacattagcaatacatatggagagacacctgaatggattacacgacaaacgcaactacaacatagttttgaggatagtacctttgaattatcacaaatggtgcaatgggcatttgaccatgaagtattagatgatagtgaaatagcatttcattatgcacaattagcagatatagatagtaatgctgcagcgtttttaaagagtaattgccaagcaaaatatgtaaaagattgtgggaccatggcacggcattacaaacgagcacaaagaaaatcattatccatgtcagcctggataaggtatagatgtgatagagcaaaggatggaggcaactggagagaaattgctaaatttttaagatatcaaggtgtaaactttatgtcctttattcaaatgtttaaacagtttttaaaaggaacaccaaaacacaattgcatagttatatatggcccaccaaacacaggcaagtcattatttgcaatgagcctaatgaagtttatgcaagggtccattatttcatatgtaaactctggtagtcatttttggttacagccactagaggatgctaaaatagcattgttagatgatgctacgtatgggtgttggacatatattgatcagtatttaagaaactttttagatggtaatccatgtagtatagatagaaaacataggagtttaatacaattagtatgtccaccattactaataacgtcaaacataaatccacaagaggatgcaaacctaatgtatttacatacaagggtaacagtattaaagtttttaaatacatttccatttgataacaatgggaatgctgtgtatacattgaatgatgaaaattggaaaaattttttttccaccacatggtccagattagatttggaggaggaagaggacaaagaaaatggagaccctatgccaccgtttaaatgtgtgccaggagaaaatactagactgttatgaactggacagtgataaattagtagatcaaattaactattggacattgttacgatatgaagctgctatgttttatgcagcacgggaaagaaacttacaaacaatcaatcaccaggtagtaccagcaacaacagtatcaaaacaaaaggcctgtcaagcaattgaaatgcacatggccttacaatcgcttaacaaatcagactataacatggaaccatggacaatgcgggagacatgttatgaactatggtgtgtggctcccaagcaatgtttcaaaaaggggggcataactgtaacagttatatttgatggaaataaggacaatgcaatggactatacaagctggaaatttgtatatatatatgataatgataagtgggtaaagacagatggaaatgtggactatacgggtatatattacactgtaaattcaaaaaaagagtattatgtacagtttaaagatgaagccaaaagatatggggcacaacagtgggaggtctatatgtatggtactgtaataacatgtcctgaatatgtatctagtacctgcagcgacgcgttatccactactacaactgttgaacaactatcaaacaccccaacgaccaatccccttaccacctgcgtgggcgccaaagaagcccagacacaacagcgaaaacgacagcgacttactgagcccgactcctccacaatctccccactgtccgtggacaatacagacaaccaaatacactgtggaagtggaagcactaacactggagggcaccaaagtgcaactcagactgcgtttatagtgcatttaaaaggtgatacaaattgtttaaaatgttttagatacagatttacaaaacacaaagggttatataaaaacgtatcctcaacctggcattggaccagtaatactaaaacaggcattgttaccattgtgtttgacagtgcacatcaacgggaaacatttataaaaaccattaaagtacccccaagtgtaacactgtcattgggaattatgacactgtaactactgtaatatatgtattgtacatatatactgtcacaagccaatatgtgctgctaagtgtatagacatatagtaaccattgcagtgtttattattttgctatttgtgctttgcgtgtgtgtgtgtcttgtgttgtgttgtttgttgccgctactgctgtcccaatacgtgtttgcagctgccttattattaattttatgtttttggtttgttgttgcaacatcccaattaactacattttttgtatatttgatttttttttacttaccttgtttacttttacatctatatacatttttacttttgcaataaacttgttatatttttgtgattaaatatggtggctacacgtgcacggcgtcggaagcgggcatctgtaacacaattatattctacatgcaaagctgctggtacatgtcctcctgatgttgtgaataaggttgaaggtactacattggccgataaaatattacagtggagtgggttaggtatatttttgggtggcctaggtattggtactgggtctggatctggggggcgtactggatatatccctttaggtggtgggggtcgcccaggcgtggtggatattgctcctgcaaggccacctattataattgaacctatagcacctactgaaccttctatagtaaatttggttgaagactctagtattattcagtctgggtctcctatacctacctttactggtaccgatggctttgaaattacttcatcttccacaacaacccctgctgtgttggacataaccccatctgctggtactgtacatgtttccagtactaacattgaaaatcctttatatattgaacctccatccattgaggctccacaatctggagaagtgtcaggacatatatttactagtacacttacttctggtactcatgggtatgaagaaatacctatggaagtgtttgcatccaatgtcagtactggtactgaacctattagcagcacacctactccaggggttagtcgcatagctgctccccgcttgtatagtaagtcttacacacaagttaaagttacaaatcctgattttattagtaagccatccacatttgttacatttaataatcctgcttttgagcctattgacacatccataacttttgaggaacctgatgctgttgcacctgatcctgattttctggatattattaaactgcaccgccctgcccttacatctcgtagaggcacagtacgctttagtaggttaggtcaaaaggccaccatgcgcactcgtagtggcaaacaaattggtgctcgtgtacattattatcatgatattagtagaattgcaccagctgatgaacttgaaatgcagcctttactttccccttctaataattatagttatgacatttatgctgatttagatgaagctgaaacaggttttatacagcccacacacaccacacctatgtcacactcctctttgtctaggcagttgccctccttatcttcatctatgtcttcatcttatgcaaatgttactattccattttcaactacatattctgttcctattcatacagggcctgatgtggtattgcccacatctcctacagtatggccttatgttccccacacttccattgacaccaagcattctattgttatactaggtggggattactatttgtggccctatacacatttactacgcaaacgccgtaaacgtataccctatttttttacagatggcattgtggcgcactaatgacagcaaggtgtatttgccacctgcacctgtgtctcgaattgtgaatacagaagaatatatcacacgcaccggcatatattactatgcaggcagttccagactaataacattaggacatccctattttccaatacctaaaacctcaacgcgtgctgctattcctaaagtatctgcatttcaatacagggtatttagggtacagttaccagatcctaacaagtttggactcccggatccaaatttatataatccagacacagataggttggtgtggggttgtgtgggcgttgaggtgggcagaggacagccccttggtgttggccttagtggtcatcccttatttaataaatatgatgacacagaaaattcacgcatagcaaatggcaatgcacaacaagatgttagagataacacatctgttgacaacaaacagactcagttatgtataataggctgtgctccacctattggggaacactggggtattggcactacatgcaaaaacacacctgtacctccaggagactgcccccccctggaacttgtatcctctgtcattcaggatggcgatatgattgatacagggtttggagctatggatttcgctgccctacaggccaccaaatcagacgtccctttggatatttcacagtctgtttgtaaatatcctgattatttaaaaatgtctgcagacacatatggtaattccatgttttttcatttacgcagggagcaaatctttgctaggcactattataataaacttggtagtgttggggaagacattcctaacgattattatattaagggtagtggtaatggccgtgaccctatagaaagttatatatactctgctactcccagtgggtctatgataacatctgattctcagatttttaataagccttattggctccaccgtgcgcagggtcacaataatggcatttgctggaacaatcagctttttattacctgtgttgatactaccagaagtacaaatttaactattagcactgccactgctgcagtttccccaccatttactccaagtaactttaagcaatatattaggcatggggaagagtatgaattgcaatttatttttcagttatgtaaaattactttaactacagaggtaatggcttatttacacacaatggatcctaccattcttgaacagtggaattttggattaacattacctccgtctgctagtttggaggatgcatacaggtttgttagaaatgcagctactagctgtcaaaaggacacccctccacaggctaagccagatcctttggccaaatataaattttgggatgttgatttaaaggaacgattttctttagatttagaccaatttgcattgggtcgcaagtttttgttgcaggttggcgtacaacgcaagcccagaccaggccttaaacgcccggcctcatcggcatcctcttcctcttcctcttcagccaaacgtaaacgtgttaaaaagtaatgtatgttagtttttgtatgcttgtgcacactgttgtatgcctgtatgtatatgtttgtgtatgtactgtatgtgtttttgtgtgtgtgtgtgttgttgttcctgtatgtatgagttatgtatgtttattattaataaactatgtggtgtgtgtgtgtgtgtttttgcatgactgcatttgtatgacatgtacgggtgtatgtgggtattacattatccccgtaggtcaagggtggtgtttcggtggcgtccctattgccctacccatttttttgcagcacaacagtttatatttatgctatttagttatactttgtagcttccattttgttacagctgcagccattttgagtgcaaccgatttcggttgctgtacttttagtatatttgccaagttttaaaccacaactgccagttgtttttggcataaaccatcagttttttatgacatagtgcatacatccgcccgcccacgccttgtacttggcgcgccttaccggcgctagtcatacaacctattagtcatttgtactttaacaattgttggcacactgttttccgccctataataatttaactgcttataggcatgtattttttggcatattttatcttactaattgcatagttggcaggtcaaatactatgtttttagtgccaagtttctatcctacttataaaccatcttactcatatgcaggtgtgctacacaaatgtgttacctaaccgatttgtgttctgcctatgcttgcaacattttttcttataacattt

SEQ ID NO: 8 - DNA, 449 bp, Human papillomavirus type 6
gcccagggacataacaatggtatttgttggggtaatcaactgtttgttactgtggtagataccacacgcagtaccaacatgacattatgtgcatccgtaactacatcttccacatacaccaattctgattataaagagtacatgcgtcatgtggaagagtatgatttacaatttatttttcaattatgtagcattacattgtctgctgaagtaatggcctatattcacacaatgaatccctctgttttggaagactggaactttgggttatcgcctcccccaaatggtacattagaagatacctataggtatgtgcagtcacaggccattacctgtcaaaagcccactcctgaaaaggaaaagccagatccctataagaaccttagtttttgggaggttaatttaaaagaaaagttttctagtgaattggatcagtatcctttgggacg

SEQ ID NO: 9 - DNA, 1506 bp, Human papillomavirus type 11
atgtggcggcctagcgacagcacagtatatgtgcctcctcctaaccctgtatccaaagttgttgccacggatgcttatgttaaacgcaccaacatattttatcatgccagcagttctagacttcttgcagtgggtcatccttattattccataaaaaaggttaacaaaactgttgtgccaaaggtgtcaggatatcaatacagagtatttaaggtggtgttaccagatcctaacaaatttgcattgcctgactcgtctctttttgatcccacaacacaacgtttggtatgggcatgcacaggcctagaggtgggccggggacagccattaggtgtgggtgtaagtggacatcctttactaaataaatatgatgatgttgaaaattcagggggttacggtggtaaccctggacaggataacagggttaatgtaggtatggattataaacaaacacaattatgcatggttggatgtgccccccctttgggcgagcattggggtaaaggtacacagtgtagtaatacatctgtacagaatggtgactgcccgcccttagaacttattaccagtgttatacaggatggcgatatggttgacacaggctttggtgctatgaattttgctgatttgcagaccaataaatcagatgttcctcttgacatatgtggcactgtatgtaaatatccagattatttacaaatggctgcagacccatatggtgatagattatttttttatctacggaaggaacaaatgtttgccagacatttttttaacagggctggtaccgtgggggaacctgtgcctgatgatcttttagttaagggtggtaacaatcgctcgtctgtagcgagtagtatatatgttcacaccccaagcggctctttggtgtcctctgaggcacaattgtttaataagccatattggctacaaaaagcccagggacataacaatggtatttgttggggtaatcatctgtttgttactgtggtagataccacacgcagtaccaacatgacattatgtgcatccgtatctaaatctgccacatacaccaattctgattataaagagtacatgcgtcatgtggaagagtttgatttacaatttatttttcaattatgtagcattacattgtctgctgaagtaatggcctatattcacacaatgaatccctctgttctcgaagactggaactttgggttatcgcctcccccaaatggtacactcgaggatacctataggtatgtgcagtcacaggccattacctgtcaaaagcccactcctgaaaaggaaaagcaagatccctataaggacatgagtttttgggaggttaatttaaaagaaaagttttctagtgaattggatcagtttcctttgggacgcaagtttttgttacaaagtggatataggggacggacctctgctcgtaccggtattaagcgccctgctgtttccaaaccctctactgcccctaaacgtaagcgcaccaaaactaaaaagtaa

SEQ ID NO: 10 - DNA, 7917 bp, Human papillomavirus type 42
cttattataaactacaatcctggctttgaaaaataagggagtaaccgaattcggttcaaccgaaaccggtacatatataaaccacccaaagtagtggtcccagttaaggcagaatgtcaggtacatctgcctcatcacagccacgcacattataccaattgtgtaaggaatttgggctgacattgcggaatttacagatttcctgcatttggtgcaaaaagcacttaacaggcgcagaggtgctcgcgtaccattttaaagatttggtagtggtgtggaggaaggactttccatatgctgcatgtgcattttgtttagaatttaattctaaaatttgtgcactgcgacactacgaaagatcagcattttggtatacagtggagaaagaaactggactacttttagaagaacaacaaattagatgtgccttgtgtcaaaagccgttatcacagagcgaaaaaaaccatcatattgatacaggtacaagatttcaatttatattgtgtcagtggacgggtcggtgtacgcattgcagaggacaatgcgtggagagacgcctaccctaaaggacattgttttgtttgacataccaacgtgtgagacacccattgacctgtattgctatgaacaattggacagctcagatgaagatgaccaagccaaacaggacatacagcgttacagaatactgtgtgtgtgtacacagtgttacaagtctgttaaactcgttgtgcagtgtacagaggcggacataagaaacctgcaacagatgcttttgggcacactggatattgtgtgtcctttgtgtgcccgcgtggagtaactgcaatggcggatgatacaggtacagaggaggggctagggtgttctggatggttttgtgtagaagctatagtagacaaaacaacagaaaatgctatttcagatgacgaggacgaaaatgtagacgatagtgggttagatcttgtggattttgtagataatagtacagtaatacatacaaagcaggtacatgcacaagccttattaaataaacaacaagcacatgcagatcaggaggcagtacaggcactaaaacgaaagctattaggcagtccatatgaaagccctgtcagtgattcacagcacagcatagacaacgaactaagtcctaggcttggcggtttaacgctatgtcgggggtcccaaggggccaaacgacgattattccagtcactggaaaatcgagacagtggatatggctattctgaagtggaagtacagcagacacaggtagaacacggacatggcgccgtacatgggactatgggtaacgggggggcagtgggtagtgaacttggggtgcaggaaaatgaagaaggtagtactacaagtacgcctacaacaagggtggtagaattacttaagtgtaagaacctgcatgcaacattgttaggtaagtttaaagaattgtttggagtgtcatttggcgatttagtaagacagtttaaaagtgacaaaagcagttgtacagactgggttattgcagcatttggggttaatcatagtattgcagaagggtttaatacattaattaaagcagattcactatatacacatatacaatggctaacctgtacgtggggcatggtgttattaatgctaattagatttaaatgtggaaaaaatcgtactacagtgtccaaaggccttagtaaattattaaacatacctacaaatcaattattaatagagccacctcggttacaaagtgtggctgccgccatatactggtttagatcaggaatatctaatgctagcattgtaaccggagacacaccagagtggattcaaagacaaacaattttagaacattgttttgcagatgcccaatttaatttaacagaaatggtgcaatgggcatatgataatgatattactgaagacagtgacattgcatatgaatatgcacaacgggcagacagggatagcaatgctgctgcatttttaaaaagtaactgccaggcaaaatatgtaaaagattgtggcgtcatgtgcagacattataaaaaagcacaaatgagacgtatgtctatgggtgcatggataaaacatagaagtgccaagataggggatagtggagattggaaacctatagtaaaatttattagatatcaacaaattgattttttagcatttatgtctgcatttaaaaagtttttacataatatacctaaaaaaagttgtttagtgttaattggtcctccaaatacaggaaaatcacagtttggaatgagtttaataaacttcttagcaggaactgtaatatcatttgtaaattcacatagccatttttggctgcagccattggacagtgcaaaaatagctatgctggatgatgcaactccaccatgttggacatatttagatatatatttaagaaatttattagatggcaatccatgcagtatagatagaaaacataaagcattaacagttgttaagtgcccaccattacttataacatcaaatacagatattagaacaaatgacaaatggaaatacctatacagcagagttagtttatttgaatttccaaatccatttccattagatacaaatggaaatcctgtatatgaattaaatgacaaaaattggaaatcattttttcaaaggttgtggtccagcttagaatttcaagaatcagaggacgaggaagactatggagagactggccaaacgtttagatgcgtgccaggaacagttgttagaactgtatgaggaaaatagtagggatttacaaaaacatattgaacattggaaatgtttacgtatggaggcagtggtattgtataaggcccgtgaaatgggctttgcaaatataggacatcaaatagtaccaacattggaaacatgtagagccaaggcccacatggcaattgaaatacacttggcattagagacattattgcagtcctcgtatggtaaagaaccatggacattgcaagaaacaagtaatgaactgtggcttacgaatcctaaaaaatgttttaaaaaacaaggacgtaccgtggaggttatatttgatggaaaacaggacaatgcaatgcattatacagcatggacatatatatatatacaaactgtgcaaggtacatggtgtaaagtacaaggacacgtttgccatgcaggactatattatattgtggaaaatatgaaacagttttattgtaattttaaagaggaggcaaaaaaatatggggtaacagaccaatgggaggtacatgatggcaatcaggtgattgtttctcctgcacccatatctagcaccacatccaccgacgcagagataccctctactggatctactaagttggtacaacaagtgtgcaccacaaacccattgcacaccacaacgtccattgacaaccaccacgcagactgtacagacggaacagcatacaacgtgcccatccaaacctcaccgccacgaaaacgatacagacagtgtggacagtcgccatcacagcacctgcagcactcaaaccccagcatccccagcatccccagcgcatccgtggaccctggattgtgtggggtcagaactaacagtgaaaactgtaacaagcgacggaaccactgtggaagtcaggctacgcctgtaattcatttacaaggtgaccctaattgcctaaaatgcctacgatttaggctaaaaagaaattgttcacatttatttacacaggtgtcatctacatggcatttaacagaaaatgattgtacacgtgacactaaaactggtataataacaatacattattatgatgaagcacaaagaaatttatttttaaatactgtaaaaataccttctgggataaaatcctgtattggatatatgtctatgttacagtttatatgattagttgtatatgtgtataaacagttataggacttcaatactgtgactccacaacgtgtgggacaaccggccagaaactgctgcttttattgtttatagttgttggtgcgtgtgttgtgtgtgtgtggattagtttacaaaattatccatatcctgtatgggcctcttgccttgctagctacctaacattggtgctattatcatggttgcaggtactaacatactttgactatttttttctatgtttaatcattcttggtattccttctgtcttactaacattactaatacatttagcaatacaataacacatattagtttaggtgtgtgtgtgtggtgtgcatgtgatttgtacatggttgtacatatataataccaattattgtttggctactattttcatttatagccacactgctgttttgcatattggtattacaaacatataaactgttaccatacgtatatacagtgctgtaaataaacttttgttatattgtgtgtacttcttttgtgctattacaatgccaccacaacggtcccgcagacgaaagcgggcctctgccacacaattatatcaaacgtgtaaggcctcagggacatgtcctccagatgttattcccaaagttgaaggaaccacattggcagataaaattttacaatggggtagtttaggcgtgttttttggggggttgggaattggcactggtgcaggtacgggtgggcgcacgggctatgtgcctctgggaacaaggcctcctgtaattgctgaaccaggacctgcagtacgcccaccaatagctgttgacaccgtggggccatctgatccttctattgtttccttattagaagagtcatcagttattgatgcaggaataacagtacctgatattacttctcatggaggttttaatattactacatctactggtgggcctgcctcaacgcctgctatattagatatctcccctcccactaatactatacgtgtcacaacaactacatctaccaatcctttatatattgatccttttacattgcagccgccattgccagcagaggttaatgggcgcctattaatatctactcctaccatcacaccccactcatatgaagaaataccaatggacacgtttgttgtatctacagatacaactaacacatttactagtactcccattcctggccctcggtcgtctgcacgcctggggttatattctagagcaacgcaacaacgtccagttactaccagtgcatttttaacatctcctgcacggttggttacttatgacaatccagcctatgaaggacttacggaggatacattagtatttgaacatccatccattcatactgcacctgaccctgatttcatggatatagttgcattgcatcgtcctatgttatcatccaaacagggtagtgtacgtgttagtagaattggacaaaggctgtctatgcagacacgtcgcgggacccgttttgggtcacgtgtacacttttttcatgaccttagccctattacacactcttcagaaactattgaattacagcctttatctgcttcttcagtatctgcagcctccaatattaatgatgggttatttgatatttatgttgatactagtgatgtaaatgttacaaataccacttcctctatacctatgcatggttttgctaccccccgtttgtccactacatctttccctacattacctagcatgtctacacattctgccaataccaccatacctttttcgtttcctgccactgtgcatgtgggccctgatttatctgttgtggaccacccatgggacagtaccccaacgtctgtaatgcctcagggtaactttgtaatggtatcaggatgggattttatattgcatcctagttatttttggcgtaggcgccgtaaacctgtaccatatttttttgcagatgtccgtgtggcggcctagtgacaacaaggtttatctacctcctcctcctgtttccaaggtggtcagcactgatgaatatgtgcaacgcaccaactacttttaccatgccagcagttctaggctattggttgttggtcacccttattactctattacaaaaaggccaaataagacatctatccccaaagtgtctggtttacagtacagagtatttagagttaggctccctgatcctaataagtttacattgcctgaaactaatttatataacccagagacacagcgcatggtgtgggcctgtgtggggctagaagtaggtcgtggacagcctttgggcgttggtattagtggccatccattattgaataagttggatgatactgaaaatgcgcctacatatggtggaggccctggtacagacaatagggaaaatgtttctatggattataaacaaacacagttgtgtttagttggctgtaaacctgccataggggagcactggggtaaaggtactgcctgtacaccacagtccaatggtgactgcccaccattagaattaaaaaatagttttattcaggatggggatatggtggatgtagggtttggggcactagattttggtgctttacaatcctccaaagctgaggtacctttggatattgtaaattcaattactaaatatcctgattacttaaaaatgtctgctgaggcctatggtgacagtatgtttttctttttaaggcgagaacaaatgtttgttcgtcatttgtttaatagggctggcgcaattggtgaacctgtacctgatgaactgtataccaaggctgctaataatgcatctggcagacataatttaggtagtagtatttattatcctacccctagtggttctatggtaacatctgatgcacaactatttaataaaccatattggttacaacaagcacaaggacacaataatggtatatgttggggaaatcagctatttttaactgtggttgatactacccgtagtactaacatgactttgtgtgccactgcaacatctggtgatacatatacagctgctaattttaaggaatatttaagacatgctgaagaatatgatgtgcaatttatatttcaattgtgtaaaataacattaactgttgaagttatgtcatatatacacaatatgaatcctaacatattagaggagtggaatgttggtgttgcaccaccaccttcaggaactttagaagatagttataggtatgtacaatcagaagctattcgctgtcaggctaaggtaacaacgccagaaaaaaaggatccttattcagacttttggttttgggaggtaaatttatctgaaaagttttctactgatttagatcaatttcctttaggtagaaagtttttactgcaggccgggttgcgtgcaaggcctaaactgtctgtaggtaaacgaaaggcgtctacagctaaatctgtttcttcagctaaacgtaagaaaacacacaaatagatgtatgtagtaatgttatgatacatatttatgttatttatttgtgtactgtgttaataaactactttttatatgttgtgtgttctccattttgttttttgtactccattttgtttctagaccgatttcggttgtatctggcctgttaccaggtgcattggccatgtttcctaacattttgcaaacctattcactttttaaatttataaatgcaatatgtgctgccaactgttttatggcacgtatgttctgccaacgtacactccctaattcctttacataacacacacgcctttgcacaggcatgtgcacaaaggttggcaaaggttagcatatctctgcagttacccatttcctttttccttttttttatgtatgagtaacttaattgttatatgtaataaaaaagcttttaggcacatattttcagtgttggcatacacatttacaagttaccttggcttaaacaagtaaagttatttgtcactgttgacacattactcatatatataatttgtttttaacatgcaggtggcaaccgaaaccggtacataaatccttcttattctttt

SEQ ID NO: 11 - DNA, 28 bp, Artificial Sequence
  synthetic oligonucleotide
  misc_feature (13)..(13): fluorescein-dT
  misc_feature (14)..(14): riboadenosine
  misc_feature (15)..(15): dabcyl-dT
actcttcctagcnnnggttcgatcaaga

SEQ ID NO: 12 - DNA, 99 bp, Artificial Sequence
  synthetic oligonucleotide
cacggatcctgacaaggatgtgtgcgttgtcgagacctgcgaccggaacactacactgtgtgggatggatttctttacagttgtgtgcagctccgtccg

SEQ ID NO: 13 - DNA, 452 bp, Human papillomavirus type 16; identical to SEQ ID NO: 1